SEQUENCE LISTING (1 sequence)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

(19) United States
(12) Patent Application Publication
(10) Pub. No.: US 2026/0166115 A1
(43) Pub. Date: Jun. 18, 2026

(54) POLYUNSATURATED FATTY ACID-BOUND ALPHA FETOPROTEIN PROMOTES IMMUNE SUPPRESSION BY ALTERING HUMAN DENDRITIC CELL METABOLISM

(71) Applicants: The Regents of the University of California, Oakland, CA (US); Parker Institute for Cancer Immunotherapy, San Francisco, CA (US)

(72) Inventors: Lisa Helene Butterfield, San Francisco, CA (US); Juraj Adamik, Pacifica, CA (US); Paul Veness Munson, Oakland, CA (US)

(73) Assignees: The Regents of the University of California, Oakland, CA (US); Parker Institute for Cancer Immunotherapy, San Francisco, CA (US)

(21) Appl. No.: 19/127,631

(22) PCT Filed: Nov. 14, 2023
(86) PCT No.: PCT/US2023/037255

§ 371 Date: May 6, 2025

Related U.S. Application Data
(60) Provisional application No. 63/427,053, filed on Nov. 21, 2022.

(51) Int. Cl. A61K 38/00; A61K 9/00; A61K 40/24; A61K 40/42; C12N 5/0784

(52) U.S. Cl. CPC A61K 38/00; A61K 9/0053; A61K 40/24; A61K 40/42; C12N 5/0639

(57) ABSTRACT

Depending on the fatty acid bound by alpha fetoprotein (AFP), the protein can have an immunosuppressive effect, allowing for treatment of inflammatory diseases, or a neutral effect, allowing for development of dendritic cell vaccines that express AFP peptides, which have use in treating and preventing tumor AFP-expressing cancers.

### CROSS-REFERENCE TO RELATED PATENT APPLICATIONS

The present patent application claims benefit of priority to U.S. Provisional Patent Application No. 63/427,053, filed Nov. 21, 2022, which is incorporated by reference for all purposes.

### BACKGROUND OF THE INVENTION

Liver cancer accounts for 8.3% of cancer deaths worldwide, making it the third leading cause of cancer mortality (Sung. H. et al. Global cancer statistics 2020: GLOBOCAN estimates of incidence and mortality worldwide for 36 cancers in 185 countries. CA. Cancer J. Clin. (2021) doi:10.3322/caac.21660). Hepatocellular carcinoma (HCC) represents 70-85% of primary liver cancers (Jemal, A. et al. Global cancer statistics. CA. Cancer J. Clin. 61, 69-90 (2011)). Important drivers of HCC rates include chronic hepatitis B (HBV) and C (HCV) infections and control of these infections has decreased HCC rates in East Asia and Southern Europe (Bertuccio, P. et al. Global trends and predictions in hepatocellular carcinoma mortality. J. Hepatol. 67, 302-309 (2017)). Unfortunately, downward trends in HBV and HCV infections are offset by increases in other HCC risk factors, including alcohol consumption, smoking, and obesity. Obesity can lead to fatty infiltration into the liver causing non-alcoholic fatty liver disease (NAFLD), leading to non-alcoholic steatohepatitis (NASH) (Review Team et al. World Gastroenterology Organisation global guidelines: Nonalcoholic fatty liver disease and nonalcoholic steatohepatitis. J Clin. Gastroenterol. 48, 467-473 (2014). In the United States, more than 1 in 3 people have some form of NAFLD, and 6 million people have NASH (Review Team et al. World Gastroenterology Organisation global guidelines: Nonalcoholic fatty liver disease and nonalcoholic steatohepatitis. J. Clin. Gastroenterol. 48, 467-473 (2014). Given HCC's lethality, coupled with the concerning rise in HCC risk factors, new therapies are urgently needed.

Treatments for patients with early stages of HCC include surgery, ablative therapies, embolization approaches, or liver transplantation (PMID: 33479224) can be effective. For the majority of patients with more advanced stages of disease, systemic therapy options have expanded in recent years to include small-molecule multikinase inhibitors, monoclonal antibodies targeting vascular endothelial growth factor (VEGF) or its receptors, and most recently, immune checkpoint inhibition (references: Llovet overview Nature Review: https://www.ncbi.nlm.nih.gov/pubmed/33479224; Kudo lenva: 29433850; Finn atezo+bev: 32786201; Abou-Alfa cabo: 29972759; Bruix rego: 27932229; Llovet sorafenib: 18650514; Zhu ramu: 30665869; Finn (2) pembro: 31790344; Yau nivo+ipi: 33001135). The combination of bevacizumab and atezolizumab, targeting VEGF and PD-L1, respectively, has emerged as a new global standard for first-line therapy based upon substantial improvement in outcomes compared to the multikinase inhibitor, sorafenib, with median overall survival of 19.2 months for the combination versus 13.4 months for sorafenib (HR 0.66, p=0.0009) (Finn, R. S. et al. Atezolizumab plus Bevacizumab in Unresectable Hepatocellular Carcinoma. N. Engl. J. Med. 382, 1894-1905 (2020) plus updated results: https://ascopubs.org/doi/abs/10.1200/JCO.2021.39.3_suppl.267). Objective responses occurred in 30% of patients treated with the combination, including 8% with complete responses, with median duration of response not reached. Other immunotherapy combinations have also shown striking improvements in rates of objective radiographic response compared to historical controls (references: PMIDs: 32716739, 33001135, 34292792), and the combination of the PD-L1 inhibitor, durvalumab, with the CTLA-4 inhibitor, tremelimumab, improved overall survival compared with sorafenib in a randomized, phase III trial (Abou-Alfa NEJM Evidence https://doi.org/i0.1056/EVIDoa2100070). (Finn, R. S. et al. Atezolizumab plus Bevacizumab in Unresectable Hepatocellular Carcinoma. N. Engl. J Med. 382, 1894-1905 (2020)). These studies demonstrate the potential for robust and durable immune responses in a subset of patients with HCC and underscore the urgent necessity to identify and address mechanisms of resistance in the majority of patients who do not achieve prolonged responses. While immunotherapies blocking exhaustion markers (PD-1, PD-L1, and CTLA-4) and/or VEGF are encouraging, there are additional barriers in vivo that limit the potency of anti-tumor immunity.

Alpha-fetoprotein (AFP) is an oncofetal glycoprotein, similar to albumin, which is expressed by the majority of HCC tumors (tAFP) and can be detected in serum as well as the tumor microenvironment. Elevated serum concentration of AFP is associated with poor prognosis across stages of HCC, and tumors with high AFP expression may represent a distinct biologic subtype associated with activation of proliferative pathways and vascular endothelial growth factor (VEGF) signaling (Bai 2017: https://doi.org/10.1038/s41598-017-12834-1; Hoshida 2012: https://doi.org/10.1053/j.seminoncol.2012.05.003; Montal 2019: https://doi.org/10.1038/s41416-019-0513-7). Like albumin, tAFP is a secreted protein that can bind multiple metabolites and enter activated lymphocytes, hepatocytes, natural killer (NK) cells, and monocytes. Since its initial discovery in an HCC patient in the 1960's (Tatarinov, I. S. [DETECTION OF EMBRYO-SPECIFIC ALPHA-GLOBULIN IN THE BLOOD SERUM OF A PATIENT WITH PRIMARY LIVER CANCER]. Vopr. Med. Khim. 10, 90-91 (1964), interest in tAFP has focused on its prognostic (Kelley, R. K. et al. Serum Alpha-fetoprotein Levels and Clinical Outcomes in the Phase III CELESTIAL Study of Cabozantinib versus Placebo in Patients with Advanced Hepatocellular Carcinoma. Clin. Cancer Res. Off J. Am. Assoc. Cancer Res. 26, 4795-4804 (2020) and diagnostic potential in HCC (Farinati, F. et al. Diagnostic and prognostic role of alpha-fetoprotein in hepatocellular carcinoma: both or neither? Am. J. Gastroenterol. 101, 524-532 (2006)), as a cancer vaccine antigen target (Butterfield, L. H. et al. Generation of human T-cell responses to an HLA-A2.1-restricted peptide epitope derived from alpha-fetoprotein. Cancer Res. 59, 3134-3142 (1999); Butterfield, L. H. et al. T-Cell Responses to HLA-A*0201 Immunodominant Peptides Derived from α-Fetoprotein in Patients with Hepatocellular Cancer. Clin. Cancer Res. 9, 5902-5908 (2003); Butterfield, L. H. et al. A phase I/II trial testing immunization of hepatocellular carcinoma patients with dendritic cells pulsed with four alpha-fetoprotein peptides. Clin. Cancer Res. Off J. Am. Assoc. Cancer Res. 12, 2817-2825 (2006)), and its immunoregulatory properties on NK cells (Vujanovic, L. et al. Tumor-Derived α-Fetoprotein Directly Drives Human Natural Killer-Cell Activation and Subsequent Cell Death. Cancer Immunol. Res. 5, 493-502 (2017)), macrophages (Lu, C. Y., Changelian, P. S. & Unanue, E. R. Alpha-fetoprotein inhibits macrophage expression of Ia antigens. J Immunol. Baltim. Md 1950 132, 1722-7 (1984); Aussel, C. & Fehlmann, M. α-Fetoprotein stimulates leukotriene synthesis in P388D1 macrophages. Cell. Immunol. 101, 415-424 (1986)), monocytes, and dendritic cells (Pardee, A. D., Shi, J. & Butterfield. L. H. Tumor-Derived α-Fetoprotein Impairs the Differentiation and T Cell Stimulatory Activity of Human Dendritic Cells. J. Immunol. 193, 5723-5732 (2014); Santos, P. M. et al. Tumor-Derived α-Fetoprotein Suppresses Fatty Acid Metabolism and Oxidative Phosphorylation in Dendritic Cells. Cancer Immunol. Res. 7, 1001-1012 (2019)) (DCs). Our group demonstrated that tAFP has more potent immunoregulatory properties than cord blood-derived “normal” AFP (nAFP) (Pardee, A. D., Shi, J. & Butterfield, L. H. Tumor-Derived α-Fetoprotein Impairs the Differentiation and T Cell Stimulatory Activity of Human Dendritic Cells. J. Immunol. 193, 5723-5732 (2014)). The molecular features of AFP that are immunoregulatory have been attributed to differences in glycosylation patterns (Vessella, R. L. et al. Evaluation of AFP glycosylation heterogeneity in cancer patients with AFP-producing tumors. Int. J Cancer 34, 309-314 (1984); Aoyagi, Y. et al. The fucosylation index of alpha-fetoprotein and its usefulness in the early diagnosis of hepatocellular carcinoma. Cancer 61, 769-774 (1988)), isoforms (Lamerz, R. AFP isoforms and their clinical significance (overview). Anticancer Res. 17, 2927-2930 (1997); Mizejewski, G. J. Alpha-fetoprotein structure and function: relevance to isoforms, epitopes, and conformational variants. Exp. Biol. Med. Maywood NJ 226, 377-408 (2001)) or isoelectric points (Burditt, L. J., Johnson, M. M., Johnson, P. J. & Williams, R. Detection of hepatocellular carcinoma-specific alpha-fetoprotein by isoelectric focusing. Cancer 74, 25-29 (1994)), and the presence of specific ligands (Pardee, A. D., Shi, J. & Butterfield, L. H. Tumor-Derived α-Fetoprotein Impairs the Differentiation and T Cell Stimulatory Activity of Human Dendritic Cells. J Immunol. 193, 5723-5732 (2014); Benassayag, C., Vallette, G., Delorme. J., Savu, L. & Nunez, E. A. High affinity of nonesterified polyunsaturated fatty acids for rat alpha-fetoprotein (AFP). Oncodevelopmental Biol. Med. J Int. Soc. Oncodevelopmental Biol. Med. 1, 27-36 (1980); Wu, J. T., Monir-Vaghefi, S. M. & Clayton, F. Human alpha-fetoprotein and albumin: differences in zinc binding. Clin. Physiol. Biochem. 5, 85-94 (1987); Pcmiyakov, S. E. et al. Human alpha-fctoprotcin as a Zn(2+)-binding protein. Tight cation binding is not accompanied by global changes in protein structure and stability. Biochim. Biophys. Acta 1586, 1-10 (2002)). In addition, our group has determined that tAFP-mediated suppression of DCs' function depends on a low molecular mass (LMM) (Pardee, A. D., Shi, J. & Butterfield, L. H. Tumor-Derived α-Fetoprotein Impairs the Differentiation and T Cell Stimulatory Activity of Human Dendritic Cells. J Immunol. 193, 5723-5732 (2014)) molecule that is neither protein nor glycan.

### BRIEF SUMMARY OF THE INVENTION

In some embodiments, a pharmaceutical composition is provided comprising (i) alpha fetoprotein (AFP) and (ii) dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ is present in a concentration at least 2, 5, 10, 20, 30, 40, 50, 60, 70, 8-0, 90, or 100-fold higher than any polyunsaturated fatty acids other than dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the AFP is tumor AFP (tAFP) or normal AFP (nAFP) or synthetic AFP.

Also provided is a method of reducing an inflammatory response in a human in need thereof. In some embodiments, the method comprises administering a composition comprising a sufficient amount of alpha fetoprotein (AFP) bound to dihomo-gamma-linolenic acid (20:3, n-6)-, arachidonic acid (20:4)-, prostaglandin E₂ (PGE₂), prostaglandin A₂ (PGA₂), prostacyclin (PGl₂), thromboxane A₂, leukotriene B₄, leukotriene C₄, or leukotriene E₄-bound alpha fetoprotein (AFP) thereby reducing the inflammatory response. In some embodiments, the dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ in the composition is present in a concentration at least 2, 5, 10, 20, 30, 40, 50, 60, 70, 8-0, 90, or 100-fold higher than any polyunsaturated fatty acids in the composition other than dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the AFP is tumor AFP (tAFP) or normal AFP (nAFP) or synthetic AFP. In some embodiments, the human has an autoimmune disease. In some embodiments, the human has Addison disease, celiac disease, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

Also provided is a method of making the pharmaceutical composition as described above of elsewhere herein. In some embodiments, the method comprises providing recombinant or purified alpha fetoprotein (AFP); and mixing the AFP with a fatty acid selected from the group consisting of dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ to generate dihomo-gamma-linolenic acid (20:3, n-6)-, arachidonic acid (20:4)-, prostaglandin E₂ (PGE₂), prostaglandin A₂ (PGA₂), prostacyclin (PGl₂), thromboxane A₂, leukotriene B₄, leukotriene C₄, or leukotriene E₄-bound AFP. In some embodiments, the mixing comprises forming a mixture of the tAFP and the fatty acid at a molar ratio of between 1:1 and 1:5. In some embodiments, the dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ is present in a concentration at least 2, 5, 10, 20, 30, 40, 50, 60, 70, 8-0, 90, or 100-fold higher than any polyunsaturated fatty acids in the mixture other than dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the AFP is tumor AFP (tAFP) or normal AFP (nAFP) or synthetic AFP.

Also provided is a method of preparing a dendritic cell vaccine. In some embodiments, the method comprises culturing dendritic cells in a culture medium comprising palmitic acid-bound tumor alpha fetoprotein (tAFP) under conditions to generate mature dendritic cells expressing tAFP-derived HLA-restricted peptides. In some embodiments, the culturing occurs in in the absence of other polyunsaturated fatty acids. In some embodiments, the culture medium comprises a zinc chelator. In some embodiments, the dendritic cells are immature dendritic cells. In some embodiments, the dendritic cells are mature dendritic cells.

Also provided is a dendritic cell vaccine comprising mature dendritic cells cultured in the presence of palmitic acid-bound alpha fetoprotein (AFP) in the absence of other polyunsaturated fatty acids, wherein the mature dendritic cells express AFP-derived HLA-restricted peptides. In some embodiments, the AFP is tumor AFP (tAFP) or normal AFP (nAFP) or synthetic AFP.

Also provided is a method of stimulating an immune response to tAFP in a human. In some embodiments, the method comprises administering to a human a sufficient amount of the dendritic cell vaccine as described above or elsewhere herein to stimulate an immune response to tAFP. In some embodiments, the human has AFP-positive liver cancer. In some embodiments, the method comprises obtaining immature dendritic cells from the human, culturing the immature dendritic cells in a culture medium comprising palmitic acid-bound tumor alpha fetoprotein (tAFP) in the absence of other polyunsaturated fatty acids under conditions to generate mature dendritic cells expressing tAFP-derived HLA-restricted peptides, and then administering the dendritic cells to the human. In some embodiments, the culture medium comprises a zinc chelator.

Also provided is a method comprising obtaining a sample from a human having liver cancer; and measuring the amount of, and identity of, polyunsaturated fatty acids bound to tumor alpha fetoprotein (tAFP) in the sample. In some embodiments, this method is used to diagnose or provide a prognosis or treatment for the human.

### BRIEF DESCRIPTION OF THE DRAWINGS

FIG. 1A-C: tAFP exposure skews DC metabolism to glucose dependency. CD14+ monocytes were isolated from healthy donors and treated with IL-4+GM-CSF in the presence of OVA, nAFP or tAFP to produce immature dendritic cells (iDCs) and in some experiments treated with IFNγ and LPS to produce mature DCs (mDCs) (A). A representative gating scheme is shown to identify live cells containing puromycin, as well as expressing markers consistent with iDCs (HLA-DR and CD206) and mDCs (CD86) (A). DCs treated with OVA (black), nAFP (blue) and tAFP (red) were clustered based on their immune parameters by tSNE, as well as the percent of live cells expressing HLA-DR, CD206, and CD86. The proportion of cells expressing HLA-DR, CD206, CD86 and ICOSLG are shown from 3-5 technical replicates from a single donor (A). Shown are 3 technical replicates of one healthy donor of a SCENITH assay to quantify the glucose dependency (black), mitochondrial dependency (blue), glycolytic capacity (red), and fatty acid and glutaminolysis (FAAO) (purple) with mDCs treated with OVA (black), nAFP (blue) or tAFP (red) (B). Glucose and concentrations of cellular supernatants and intracellular glucose are shown with three technical replicates form a single donor, as well as the intracellular glucose uptake as determined by the influx of the fluorescent glucose analogue 2NDBG from a single replicate from a single donor (C). Shown are cellular supernatants of in vitro generated DCs from 3 healthy donors performed in technical replicates, as well as correlations with glucose in the supernatant and mitochondrial size. (D). The glucose supernatant concentrations, % pAMPK, and CD36 gMFI are shown from healthy donors (N=3) treated with OVA, nAFP or tAFP. The correlation between supernatant fatty acids and CD36 levels are shown.

FIG. 2A-D: Mitochondria expression correlates with costimulatory molecule expression. Shown are puromycin histograms (a measurement of translation and a surrogate for ATP production) for OVA (black), nAFP (blue) or tAFP (red) treated DCs. tAFP treated DCs treated with oligomycin were separated into puromycin low (purple) and high (green). The expression levels of PD-L1 and CD86 are shown in the puromycin low and high DCs (A). Mitochondrial size, as measured by mitotracker was determined in OVA, nAFP, and tAFP treated DCs (B). mDCs treated with OVA, nAFP, or tAFP were characterized by mitochondrial size with high (dark red), mid/high (pink), mid/low (teal), and low (dark blue) and clustered based on immune parameters by tSNE colored by mitochondrial size or treatment condition. Shown are the expression levels of CD80 and ICOSLG based on mitochondrial size (C). Correlations between mitotracker and CD80, ICOSLG, B7-H3, and PD-L1 are shown. Differences in PD-L1 expression levels are shown between mito hi and mito lo cells color coded by treatment condition (D). All data are from one donor performed in 3 technical replicates.

FIG. 3A-D: Immune and metabolic profile by scMEP. mDC were generated in vitro in the presence of OVA, nAFP, tAFP and analyzed the immune-metabolic profile was determined by scMEP using CyTOF. Shown are the arc sin h transformed values for immune response related molecules (A), and metabolic pathway proteins (B). A schematic is shown to summarize the tAFP-induced immune-metabolic changes (C). A heatmap of various metabolic markers was generated and hierarchical clustering was performed based on marker expression and treatment condition. The red box indicates the unique clustering of the tAFP treated cells (D). Data are representative of three separate healthy donors each performed in a single replicate.

FIG. 4A-C. DC gene expression profiles. Monocytes from healthy donors (N=4) were differentiated into iDCs in the presence of OVA, nAFP, and tAFP and gene expression profiles were determined by microarray. Principle components were determined, and color coded by treatment condition. Volcano plots were generated to identify genes that were differentially enriched (>2 fold change, significant adjusted P value) and that list of genes was used in a functional enrichment analysis by g:Profiler (A). Shown are predicted upregulated and downregulated pathways between tAFP and nAFP (B). Show are differences in genes involved in glycolysis and fatty acid metabolism pathways, and a schematic summary of tAFP upregulated (red) and downregulated (blue) genes (C). Data are from 4 healthy donors each performed once.

FIG. 5A-D. tAFP bound lipids are enriched for PUFAs. Levels of zinc were quantified in iDCs treated with OVA (black), nAFP (blue) or tAFP (red) in 3 technical replicates from a single donor and are shown as histograms as wells as Zn MFIs (percent of controls) (A). Shown are the total mass of all bound fatty acids and the proportion that are saturated, monounsaturated, and polyunsaturated (PUFA) fatty acids (B). Displayed are the proportions of individual fatty acids present in CellGenix DC media, OVA, nAFP, or tAFP (C). Shown is a heat map of the proportion of individual fatty acids bound to each protein (C). Volcano plots were generated based on each protein compared to each other protein and color-coded based on the class of fatty acid: saturated (green), monounsaturated (yellow), and PUFA (red) (D). The horizontal dashed line indicates the significance threshold based on a false discovery rate of 1%. A Euler diagram demonstrates the various combinations of differentially bound fatty acids (from C), indicating which are present in the media and are unique or shared amongst the proteins (D). Error bars are based on mean+/−standard deviation. Statistical differences in the mass or proportion of saturated, monounsaturated, or polyunsaturated fatty acids were determined based on a one-way ANOVA with Tukey's multiple comparison test. Volcano plots were generated based upon unpaired t-tests using a single pooled variance; multiple comparisons were accounted for using a false discovery rate of 1% via a two-stage step-up

FIG. 6A-B. Low molar mass binding partner screening. Low molar mass ligand were removed from OVA, nAFP and tAFP (A). Fatty acids (FA) were titered onto iDCs and supernatant lactate was measured. Control levels are indicated by a black dashed line and native tAFP lactate induction indicated by a red dashed line. Levels of CD206 were also measured with black and red dashed lines indicated control and native tAFP treated cells respectively (A). Individual fatty acids were added back to high molar mass (HMM) purified OVA, nAFP and tAFP proteins and supernatant lactate and CD206 levels were measured at the iDC and mDC stage. CD206 gMFI of the HIMM+FA were normalized to the HIMM only control. The red dashed line indicated the level of suppression seen with native tAFP (B). The red asterisks indicate the fatty acids most significantly associated with a decrease in CD206. Also shown is a schematic of their role in fatty acid metabolism. Data were performed with 1-3 technical replicates from one healthy donor.

FIG. 7A-K. HCC patient monocytes and DCs have a dysregulated immunometabolism. PBMCs were isolated from hepatocellular carcinoma (HCC) patients for SCENITH analysis. Shown are the % glycolytic capacity (A) and % FAAO (B) in multiple immune cell subsets (A,B). The total SCENITH metabolic profiles are shown for classic monocytes (C) and HLA-DR+ cells (D), with healthy donors (HD, in black) and HCC (in red), statistically significant differences are indicated with an asterisk. Levels of ILT3 are indicated on classical monocytes (cMo, E) and HLA-DR positive cells (F). Additionally, shown are CD206 and PD-L1 expression levels on HILA-DR+ cells (G, H). Correlations between ILT3 (I), CD206 (J), and PD-L1 (K) are shown with % FAAO in healthy donors (black circles) and HCC patients (red squares). Data are presentative from 3 healthy donors and 8 HCC patients.

FIG. 8 acts as a graphical abstract for aspects described herein. Dendritic Cells (DC) (left) versus tAFP-treated DC (right) are shown. DCs normally express antigen presentation and costimulatory molecules, as well as cytokines to induce robust T cell responses. These functions are supported by a balanced cellular metabolism including glycolysis and oxidative phosphorylation (OxPhos). In contrast, tAFP-treated DCs exhibit highly skewed glucose uptake and glycolytic metabolism, reduced fatty acid uptake and reduced OxPhos, leading to increased lactate secretion and a suppressed functional profile.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used in the specification and the appended claims, the singular forms “a”, “an”, and “the” include plural referents unless the context clearly dictates otherwise.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “effective amount,” “effective dose,” “therapeutically effective amount,” etc. refer to that amount of the therapeutic agent sufficient to ameliorate a disorder. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

“Pharmaceutically acceptable carrier” and “pharmaceutically acceptable excipient” are used interchangeably and refer to a substance or compound that aids or facilitates preparation, storage, administration, delivery, effectiveness, absorption by a subject, or any other feature of the composition for its intended use or purpose. Such pharmaceutically acceptable carrier is not biologically, or otherwise, undesirable and can be included in the compositions described herein without causing a significant adverse toxicological effect on the subject or interacting in a deleterious manner with the other components of the pharmaceutical composition.

As used herein, the term “administering”, “administration”, or “administer” means delivering the pharmaceutical composition as described herein to a target cell or a subject (e.g., a human). The pharmaceutical compositions described herein are designed for delivery to subjects in need thereof by any suitable route or a combination of different routes. In some embodiments, pharmaceutical compositions are administered intravenously.

The phrase “substantially identical,” used in the context of two nucleic acids or polypeptides, refers to a sequence that has at least 50% sequence identity with a reference sequence (e.g., SEQ ID NO: 1). Alternatively, percent identity can be any integer from 50% to 100%. Some embodiments include at least: 50%, 55%, 60%, 65% 70%, 75%, 80%, 85%⁰, 90%, 91%, 92%, 93%, 94%, 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### DETAILED DESCRIPTION OF THE INVENTION

It has been discovered that alpha fetoprotein's (AFP) effect on dendritic cells (DCs) depends on which fatty acid is associated with AFP. For example, the saturated fatty acid 16:0 (palmitic acid) promoted DC differentiation. Palmitic acid was unique among the fatty acids tested to increase CD206 expression (a marker for dendritic cell activation) and decrease lactate secretion in dendritic cells whereas most polyunsaturated fatty acids in association with tumor alpha fetoprotein (tAFP) inhibited CD206 expression. This surprising result has several implications for use of AFP. Notably, one can prepare a dendritic cell vaccine by culturing the dendritic cells with palmitic acid-bound alpha fetoprotein (AFP), thereby avoiding suppression of dendritic cells as occurs when AFP is associated with other fatty acids, and allowing for generation of dendritic cells expressing AFP-derived HLA-restricted peptides. Such cells have use as vaccines, for example.

Moreover, it has been discovered that when AFP is associated with certain polyunsaturated fatty acids, AFP has an immunosuppressive effect, for example significantly reducing CD206 expression and inhibiting dendritic cell differentiation. In view of these findings, one can use AFP associated with one or more of the identified polyunsaturated fatty acids (e.g., dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄) as an immunosuppressive pharmaceutical, for example for use in treating autoimmune disease.

In any of the embodiments described herein (for example used in the context of vaccine generation or, separately, as an anti-inflammatory as described here), any AFP protein, or a fatty-acid binding portion thereof, can be associated with the fatty acids described herein for their association with the activities described above). The human AFP protein has the amino acid sequence:

| (SEQ ID NO: 1) | |
|---|---|
| 1 | mkwvesifli flinftesrt lhrneygias ildsyqctae isladlatif faqfvqeaty |
| 61 | kevskmvkda ltaiekptgd eqssgclenq lpafleelch ekeilekygh sdccsqseeg |
| 121 | rhncflahkk ptpasiplfq vpepvtscea yeedretfmn kfiyeiarrh pflyaptill |
| 181 | waarydkiip scckaenave cfqtkaatvt kelresslln qhacavmknf gtrtfqaitv |
| 241 | tklsqkftkv nfteiqklvl dvahvhehcc rgdvldclqd gekimsyics qqdtlsnkit |
| 301 | eccklttler gqciihaend ekpeglspnl nrflgdrdfn qfssgeknif lasfvheysr |
| 361 | rhpqlavsvi lrvakgyqel lekcfqtenp lecqdkgeee lqkyiqesqa lakrscglfq |
| 421 | klgeyylqna flvaytkkap qltsselmai trkmaataat ccqlsedkll acgegaadii |
| 481 | ighlcirhem tpvnpgvgqc ctssyanrrp cfsslvvdet yvppafsddk fifhkdlcqa |
| 541 | qgvalqtmkq eflinlvkqk pqiteeqlea viadfsglle kccqgqeqev cfaeegqkli |
| 601 | sktraalgv |

In some embodiments, the AFP is normal AFP (nAFP). In some embodiments the AFP is tumor-derived AFP (tAFP). In some embodiments the AFP is synthetic AFP, which therefore may have different glycosylation from nAFP or tAFP or indeed no glycosylation at all depending on how AFP is synthesized. nAFP and tAFP have the same amino acid sequence (above) but differ by their expression patterns and in some embodiments by their glycosylation. Differences in glycosylation between tAFP and nAFP have been described by, e.g., Smith C. J., A. Ajdukiewicz, P. C. Kelleher. 1983. Ann. N. Y. Acad. Sci. 417: 69-74. As also noted in Santos, et al. Cancer Immunol Res (2019) 7 (6): 1001-1012, tumor-derived AFP carries a different glycosylation profile than does AFP derived from normal cord blood (nAFP). For example, Santos et al, reports nAFP contains <5% of the fucosylated variant AFP-L3, whereas AFP in serum of patient with HCC is >80% AFP-L3. In some embodiments, the AFP polypeptide comprises an amino acid sequence at least 80%, 95%, 90%, 95%, 98%, 99% or is 100% identical to SEQ ID NO:1.

Moreover, in some embodiments, the AFP is not a full-length AFP and instead only comprises a fatty acid binding portion or domain thereof. AFP has been described as having three domains (Domain I: amino acids 1-230; Domain II: amino acids 230-400; and Domain III: amino acids 400-609). Portions of AFP have been described to bind fatty acids. See, e.g., Nishihira et al., Biochem Biophys Res Commun. 1993 Nov. 15; 196(3):1049-57, describing amino acids 210-227 of AFP binding fatty acids.

AFP can be obtained for example by isolation (e.g., purification) of an AFP protein from a source of AFP, or alternatively AFP can be produced recombinantly (which can also comprise purification following AFP production by a recombinant cell). Sources of AFP can include, but are not limited to, tumor cells (a source for tAFP) and cord-blood (a source of nAFP). Recombinant production of AFP can be performed in any suitable prokaryotic (for example, E. coli) or eukaryotic (for example yeast, mammalian or insect cells) expression system or alternatively in a cell-free expression system.

Any of the AFP polypeptides (e.g., recombinant AFP or portion thereof) described herein can be provided as a fusion protein with any of a variety of heterologous amino acid sequences. Fusion partner sequences can include, but are not limited to, amino acid tags, non-L (e.g., D-) amino acids or other amino acid mimetics to extend in vivo half-life and/or protease resistance, targeting sequences or other sequences. A fusion domain or a fragment thereof may be selected so as to confer a desired property. For example, some fusion domains are particularly useful for isolation of the fusion proteins by affinity chromatography. Exemplary fusion domains include, but are not limited to, polyhistidine, Glu-Glu, glutathione S transferase (GST), thioredoxin, protein A, protein G, an immunoglobulin heavy chain constant region (Fc), maltose binding protein (MBP), and/or human serum albumin (HSA). In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, a protein is fused with a domain that stabilizes the protein in vivo. By “stabilizing” is meant anything that increases the life time of the protein in the circulating blood, regardless of whether this is because of decreased destruction, decreased clearance by the kidney, or other pharmacokinetic effect. Fusions with the Fc portion of subtypes IgG1 or IgG2a immunoglobulin are known to confer desirable pharmacokinetic properties on a wide range of proteins. See, e.g., US Patent Publication No. 2014/056879. Certain mutations of these Fc portions of these IgGs confer even better pharmacokinetic properties. Generation of mutated variants of the human form of the MHC class I-related receptor, FcRn, with increased affinity for mouse immunoglobulin G, as described in Zhou J, Johnson J E, Ghetie V, Ober R J, Ward E S. J Mol Biol. 2003 Sep. 26; 332(4):901-13. Likewise, fusions to human serum albumin can confer desirable properties. Other types of fusion domains that may be selected include multimerizing (e.g., dimerizing, tetramerizing) domains and functional domains (that confer an additional biological function, as desired). Fusions may be constructed such that the heterologous peptide is fused at the amino terminus of a polypeptide and/or at the carboxyl terminus of a polypeptide.

One can associate a particular fatty acid with an AFP protein simply by incubating the two together, allowing for the AFP protein to bind the fatty acid. The binding of the fatty acid to the AFP is non-covalent. By incubating the AFP protein in the presence of only one species of fatty acid, or at least at a relatively high concentration compared to the concentration of other fatty acids present, and compared to the concentration of AFP protein, one can generate a population of AFP binding essentially one species of fatty acid. In some embodiments, the molar amount of the fatty acid is greater than the molar amount of the AFP. For example, in some embodiments, the molar ratio of fatty acid:AFP is 2:1 to 10:1, though other ratios can also be used. In some embodiments, a population of AFP protein can be bound in which at least 50, 60, 70, 80, 90, or 95% of the AFP bound to a fatty acid binds to the particular fatty acid species (for example, palmitic acid, dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄).

In some embodiments, methods of making dendritic cell vaccines, as well as the resulting vaccines, and methods of inducing an immune response using the vaccines are provided. As explained in the Examples, palmitic acid-bound AFP increased CD206 expression (a marker for dendritic cell activation) and decreased lactate secretion in dendritic cells whereas most polyunsaturated fatty acids in association with tAFP inhibited CD206 expression. Thus, by incubating dendritic cells with palmitic acid-bound AFP, especially in the absence of other fatty acids in the culture medium, one will improve generation of effective dendritic vaccines expressing AFP-derived HLA-restricted peptides, at least in part by avoiding the immune-suppressive effects of other fatty acids that may associate with AFP. In some embodiments, there is at least 100- or 1000-fold more palmitic acid-bound AFP compared to other fatty acid-bound AFP in a mixture that is subsequently used to label dendritic cells. Palmitic acid is a saturated long-chain fatty acid with a 16-carbon backbone.

Dendritic cells are provided, for example, for use in generation of dendritic cell vaccines that can be introduced into a human individual to stimulate an immune response, e.g., a cellular immune response, for example a T-cell response, against AFP (for example tAFP) or tumor cells expressing tAFP. Dendritic cells can be obtained from any human source. In some embodiments, the dendritic cell vaccines are autologous to the ultimate recipient, meaning dendritic cells, or precursor cells thereof (for example but not limited to peripheral blood mononuclear cells, monocytes or other myeloid progenitor cells), are obtained from a human individual, optionally induced to differentiate into dendritic cells, cultured as described herein to express AFP-derived HLA-restricted peptides, and then introduced into the same human individual. Examples of methods of differentiating precursor cells into dendritic cells are described in, e.g., U.S. Patent Publication No. 2021/0139852. Alternatively, the dendritic cells are allogenic to the ultimate recipient, meaning the dendritic cells, or precursor cells thereof, are obtained from a different human compared to the recipient of the vaccine. Dendritic cells obtained from an individual can be mature (e.g., expressing high levels of MHC class I (HLA-ABC) and class II (HLA-DR/DP/DQ), CD80, CD86, CD40, CD83, CCR7, low CD14, expressing IL-12p70 and lack typical lineage markers CD3, CD19/20 and CD56) or immature (e.g., lower MHC class I and II (HLA-DR), CD80, CD86, CD40, no CD83, no CCR7, positive for CD14, expressing no to minimal IL-12p70) dendritic cells. Immature dendritic cells can be converted by any maturation protocol, for example, but not limited to culturing immature cells with a sufficient amount of LPS and interferon gamma.

In some embodiments, precursor cells are obtained from a human individual and then induced to differentiate into dendritic cells. For example, in some embodiments, pluripotent or multipotent precursor cells can be obtained from a human donor. In some embodiments, cells from the donor are converted to induced pluripotent stem cells (iPSCs), which are then differentiated into dendritic cells. In some embodiments, CD34+ stem cells are obtained and differentiated into dendritic cells. Examples of precursor cells differentiated into dendritic cells for use as vaccines are described in, e.g., WO 2006/020889. In some embodiments, the dendritic cells comprise, or are enriched for, a dendritic cell subpopulation, for example for myeloid dendritic cells, plasmacytoid dendritic cells, or CD14+ dendritic cells, e.g., as described in Collin, et al., Immunology. 2013 September; 140(1): 22-30.

Immature or mature dendritic cells can be cultured in culture media containing a sufficient concentration of palmitic acid-bound AFP such that the cells uptake the AFP and express HLA-restricted AFP peptides on the cells (for example, acting as antigen present cells to present AFP peptides). In some embodiments, the palmitic acid-bound AFP is incubated in the medium, which is optionally pre-warmed and mixed with the dendritic cells at 37° C. and incubated for a period of time, for example on hour. Various culture conditions for dendritic cells can be found in, e.g., U.S. Patent Publication No. 2021/0139852 and PCT Publication No. WO2006/020889, as well as in the Example below.

Optionally, the dendritic cells cultured with palmitic acid-bound AFP are cultured in a medium comprising a zinc chelator or otherwise having reduced zinc ions. An exemplary zinc chelators can include, for example, but without limitation, N,N,N′,N′-tetrakis(2-pyridinylmethyl)-1,2-ethanediamine (TPEN), 6-methoxy-8-p-toluenesulfonamido-quinoline (TSQ). See also Rapford and Lippard, Current Opinion in Chemical Biology, Volume 17, Issue 2, April 2013, Pages 129-136.

Once prepared, the dendritic cells expressing HLA-restricted AFP peptides, optionally formulated in a pharmaceutically-acceptable formulation, can be administered to a human to induce an immune response against AFP. In some embodiments, the human has cancer, e.g., a tAFP-expressing cancer. In some embodiments, the human has tAFP-expressing liver cancer. In embodiments in which the dendritic cells are allogeneic, HLA matching can be performed to select dendritic cells that have reduced or no HLA-mismatching to avoid graft-host interactions.

Dendritic cell preparations can be stored after preparation to be used later for therapeutic administration or further processing. Methods of cryopreserving dendritic cells both before and after loading are described in PCT publication WO 02/16560.

As noted above, another discovery described herein is that AFP bound to certain unsaturated fatty acids (e.g., dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄) were found to have anti-inflammatory activity and therefore are useful for treating or ameliorating inflammatory diseases, such as but not limited to autoimmune diseases. Thus, by administering a sufficient amount of AFP bound by one of these unsaturated fatty acids, one can attain an anti-inflammatory effect.

As described in the Examples, dendritic cells contacted with AFP bound to dihomo-gamma-linolenic acid (20:3, n-6) or arachidonic acid (20:4) showed significantly reduced CD206 expression. Moreover, in metabolism of ω-6 fatty acids, 20:3 N6 is converted to 20:4, which can then be further converted into a variety of molecules by COX and LOX enzymes. Taken together, these data indicate that any of these polyunsaturated fatty acids bound to AFP trigger inhibition of DC differentiation.

Accordingly, in some embodiments, compositions are provided comprising (i) alpha fetoprotein (AFP) bound to (ii) dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. These compositions can be formed by incubating AFP with the unsaturated fatty acid in question. Ideally, while incubating the AFP with the target unsaturated fatty acid, the amount of other free fatty acids will be limited such that at least a majority (e.g., at least 50, 60, 70, 80, 90, 95, 98, or 99%) of the AFP bound to a fatty acid is bound to one of dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the molar ratio of unsaturated fatty acid to AFP during incubation of the AFP with the fatty acid will be more than one, to increase the portion of AFP that is bound to a fatty acid. For example, in some embodiments, the AFP is incubated at a molar ratio of unsaturated fatty acid:AFP of at least 1, 2, 3, 4, 5, 6, 7, 8, 9, 10, e.g., from 2-10. In some embodiments, the incubation medium, and/or the resulting AFP composition, will substantially lack polyunsaturated fatty acids other than dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. “Substantially lacks” as used herein means that less than 5, 4, 3, 2, or 1% of the free unsaturated fatty acids present will be an unsaturated fatty acid other than dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄.

Also provided are pharmaceutical compositions comprising (i) alpha fetoprotein (AFP) and (ii) dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. The AFP can be for example tAFP or nAFP.

Pharmaceutical compositions comprising AFP and a fatty acid bound thereto as described herein or a dendritic cell expressing AFP peptides as described herein can include one or more pharmaceutically acceptable carriers. Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions may be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical composition may be formed in a unit dose form as needed. The amount of active component, e.g., AFP bound to a fatty acid or together cocultured with a dendritic cell as described herein, included in the pharmaceutical preparations is such that a suitable dose within the designated range is provided (e.g., a dose within the range of 0.01-500 mg/kg of body weight).

The pharmaceutical compositions may be administered in a manner compatible with the dosage formulation and in such amount as is therapeutically effective to result in an improvement or remediation of the symptoms. The pharmaceutical compositions may be administered in a variety of dosage forms, e.g., subcutaneous dosage forms, intravenous dosage forms, and oral dosage forms (e.g., ingestible solutions, drug release capsules). Pharmaceutical compositions containing the active ingredient (e.g., an antibody as described herein) may be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines.

Also provided are methods of treating or preventing or ameliorating one or more inflammatory disease in a human by administering an effective amount of AFP bound to dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄. In some embodiments, the human has an autoimmune disease. Exemplary autoimmune diseases can include but are not limited to Addison disease, celiac disease, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

In yet another embodiment, in view of the varying immune effects of fatty acids, when bound to AFP, one can obtain a sample comprising AFP from a human and assay the AFP to determine the identity of fatty acids bound to the AFP. For example, AFP-positive liver cancer samples can be assayed for the fatty acids associated with tAFP from the sample. As noted above, certain fatty acids when bound to AFP, for example, dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ are immunosuppressive and can therefore indicate a worse outcome for a cancer patient in that the tumor cells promote an immunosuppressive environment around a tumor, thereby preventing or inhibiting the human's immune system from attacking the tumor. Humans having a cancer that expresses tAFP bound to dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄ can subsequently be treated to reduce or suppress the immunosuppressive effects of the tAFP in the tumor.

### EXAMPLE

Here, utilizing novel single cell methods and lipid profiling in both in vitro models and in vivo human HCC patient blood samples, we have determined that tAFP uptake by DC causes reduced fatty acid uptake and metabolism and a switch to glycolysis accompanied by increased glucose uptake and lactate secretion. This metabolic skewing is accompanied by a shift in immune phenotype, with reduced costimulatory molecule expression and increased DC CD14 and PD-L1 expression. For the first time, we identify differences in the ligand composition between nAFP and tAFP and show that these fatty acids are essential for the immunoregulatory features of tAFP. These findings have important implications for understanding how AFP+ HCC limits innate immune responses, identifying strategies to improve DC function in vivo, and development of more potent DC vaccines.

Results—tAFP Induces Immuno-Metabolic Dysregulation of DCs

To determine the mechanism of immune suppression induced by AFP, we performed immune and metabolic profiling human DC Previously, we demonstrated using population-based assays that tAFP decreases the differentiation monocytic DCs. and reduces their T-cell stimulatory potential (Pardee, A. D., Shi, J. & Butterfield, L. H. Tumor-Derived α-Fetoprotein Impairs the Differentiation and T Cell Stimulatory Activity of Human Dendritic Cells. J. Immunol. 193, 5723-5732 (2014)). We demonstrated that tAFP limited DC HLA-DR and CD206 expression with a trend for reduced CD86. Furthermore, the Boolean analysis revealed a decreased co-expression of multiple activation markers (HLA-DR, CD206, CD86, and ICOSLG/CD275) among tAFP-treated DC.

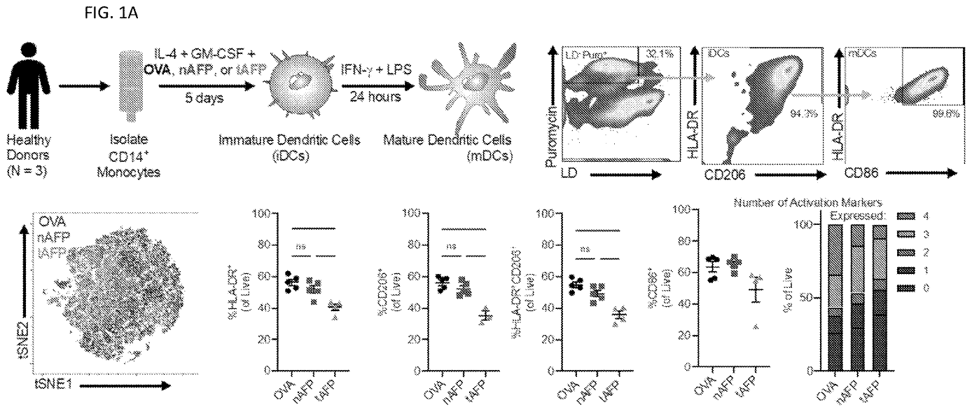

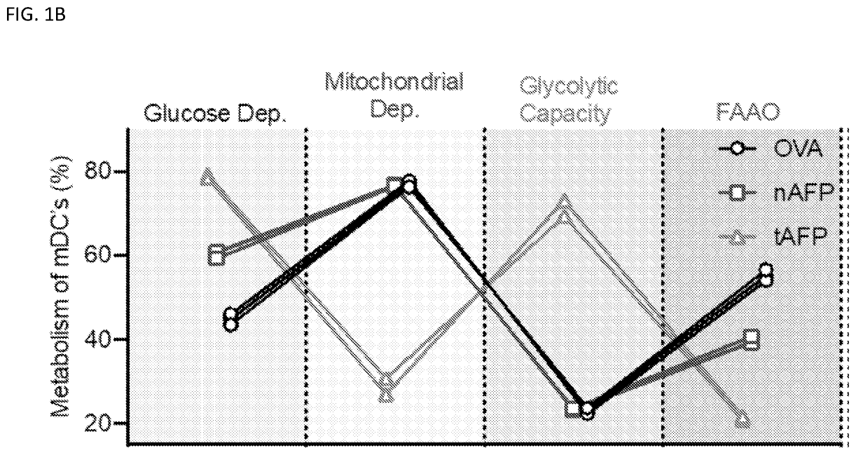

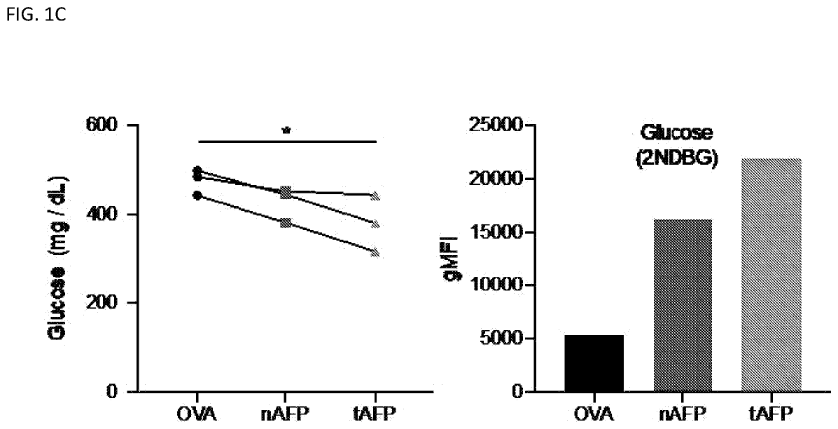

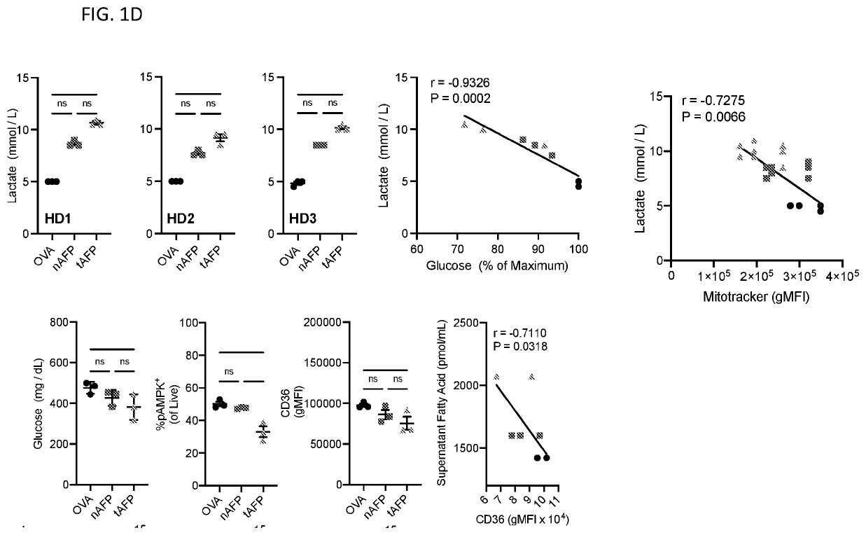

To understand the immuno-metabolic impact of tAFP on DCs at the single cell level, we utilized the recently described single-cell energetic metabolism by profiling translation inhibition (SCENITH) assay (Arguello. R. J. et al. SCENITH: A Flow Cytometry-Based Method to Functionally Profile Energy Metabolism with Single-Cell Resolution. Cell Metab. 32, 1063-1075.e7 (2020)). Ovalbumin (OVA, negative control), nAFP, and tAFP-treated DCs were generated in vitro (FIG. 1A). Viable cells (LD⁻) actively translating RNA into protein (Puro⁺) were analyzed that expressed cell surface molecules associated with mDCs (HLA-DR⁺, CD206⁺, CD86⁺, (previously shown to be representative of many common DC phenotypic markers (#22)). To assess the broad immuno-metabolic state of the cells, a tSNE analysis was performed on all parameters that indicated tAFP-treated cells tended to cluster separately from nAFP or OVA treated mDCs. The calculated metabolic profiles are shown for glucose dependency, mitochondrial dependency, glycolytic capacity, and fatty acid and glutaminolysis (FAAO) (FIG. 1B). Even among mDC with strong expression of activation markers (HLA-DR+, CD206⁺, CD86⁺), there was a dramatic increase in glycolysis and a reduction in mitochondrial dependency and FAAO in tAFP-treated DCs. Consistent with a greater reliance on glycolysis, tAFP-treated cells had significantly less glucose in the culture supernatants at day 6 (FIG. 1C) with an increase of intracellular glucose. This correlated with a decreased expression of pAMPK, which can inhibit glycolysis. In conjunction with a decrease in FAAO, a decline in expression of the fatty acid transporter CD36 was detected (FIG. 1D). Similarly, free fatty acids in the culture supernatants at day 6 were inversely correlated (r=−0.7110, P=0.0318) with the expression of CD36. Taken together, these data indicate that tAFP-treated DCs rely on glycolysis and have a decreased ability to take up and oxidize fatty acids.

In agreement with decreased mitochondrial capacity by SCENITH (FIG. 1B), we previously confirmed decreased mitochondrial mass in tAFP-treated DCs (Santos, P. M. et al. Tumor-Derived α-Fetoprotein Suppresses Fatty Acid Metabolism and Oxidative Phosphorylation in Dendritic Cells. Cancer Immunol. Res. 7, 1001-1012 (2019)). With decreased mitochondrial activity and the DC reliance on glycolysis, we investigated the potential release of lactate. Given the immunoregulatory functions of lactate, we determined if tAFP could promote lactate secretion by DC. Lactate was measured in the media of OVA, nAFP, and tAFP treated DCs (FIG. 1D). In all HDs, tAFP-treated DC secreted the most lactate at approximately twice the concentration of OVA-treated DCs, which may in part explain tAFP-treated DCs having a diminished capacity to stimulate T cells (Pardee, A. D., Shi, J. & Butterfield, L. H. Tumor-Derived α-Fetoprotein Impairs the Differentiation and T Cell Stimulatory Activity of Human Dendritic Cells. J. Immunol. 193, 5723-5732 (2014)). The increased concentrations of lactate inversely correlated with glucose in the supernatant (r=−0.9326, P=0.0002), suggesting this build-up of lactate results from increased reliance on glycolysis, as opposed to oxidative phosphorylation or FAAO, for the production of ATP.

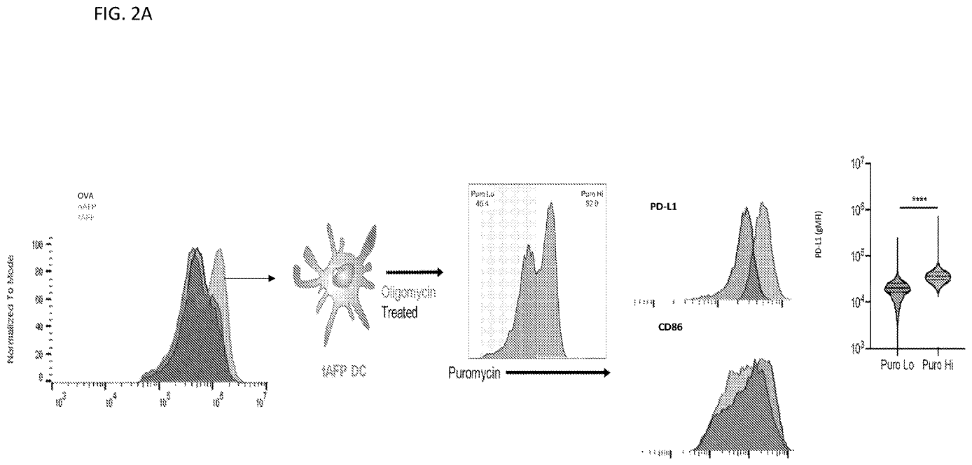

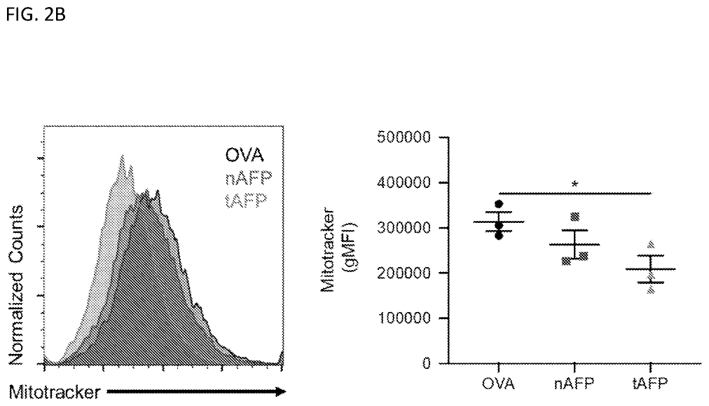

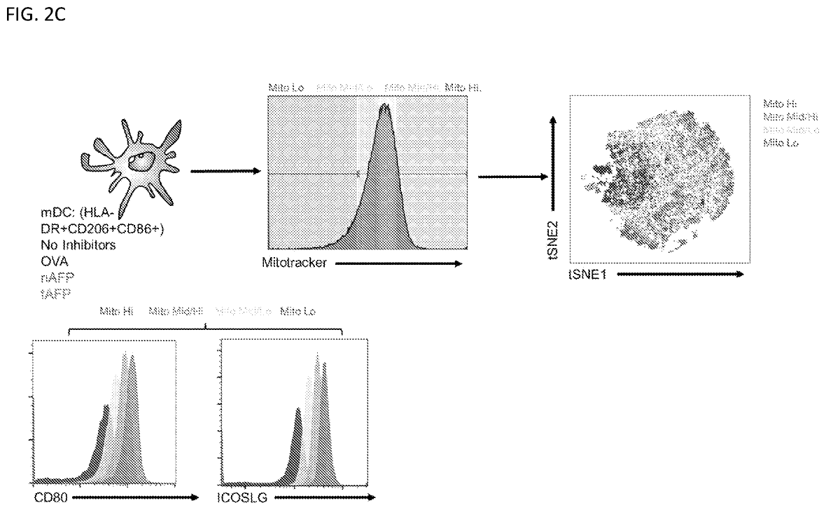

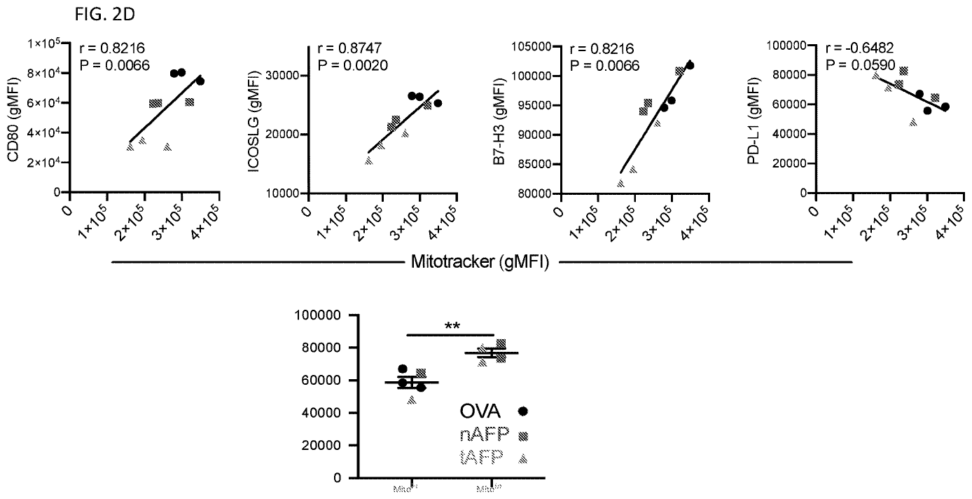

Given that tAFP induced both immune and metabolic changes, we examined connections between costimulatory markers and metabolic state. Cells were gated based on the relative mitochondrial mass (FIG. 2). As mitochondrial size decreased, the cells coalesced around a single cluster. To better understand the impact of altered mitochondrial load on the expression of key costimulatory molecules, we determined the relative expression of activation markers (i.e., CD80 and ICOSLG) based on mitochondrial size. We observed strong positive correlations between mitochondrial size and the expression of CD80 (r=0.8216, P=0.0066), ICOSLG (r=0.8747, P=0.0020), and B7-H3/CD276 (r=0.8216, P=0.0066) (FIG. 2C). While there was a trend towards a negative correlation between mitochondrial size and PD-L1/CD274 (r=−0.6482, P=0.0590), this reached statistical significance when directly comparing PD-L1 levels between Mito^{Hi} VS. Mito^{Lo} cells (FIG. 2D). These findings indicate that decreased mitochondrial mass skews the cells towards glycolysis and is also associated with weaker expression of activating ligands (CD80, ICOSLG, B7-H3) and increased expression of inhibitory ligands (PD-L1).

### Single Cell Phenotypic Profiling

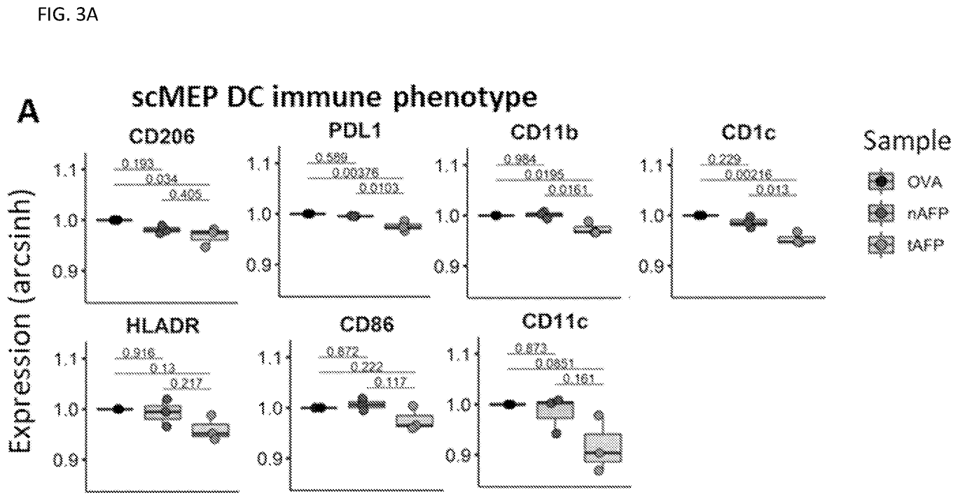

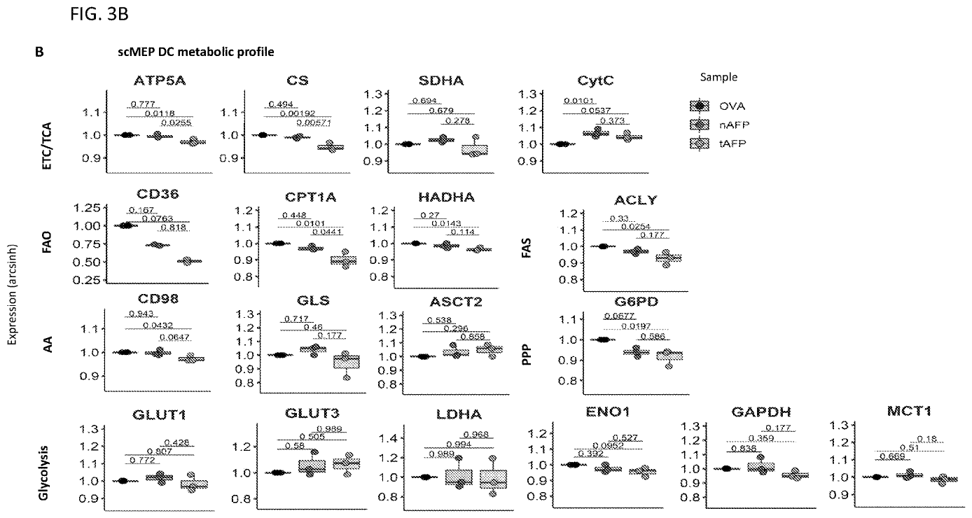

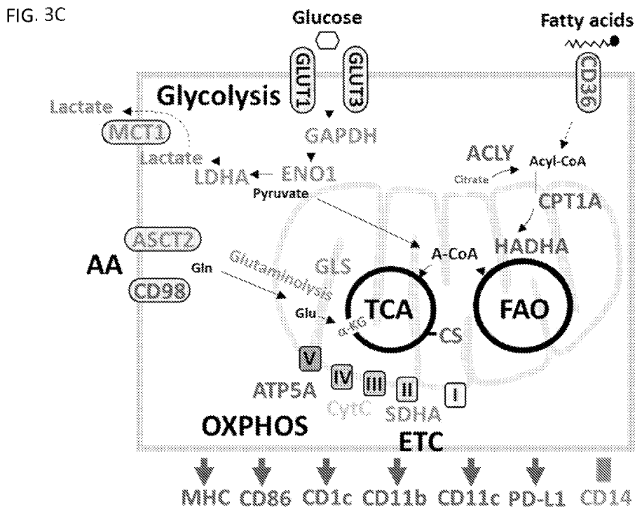

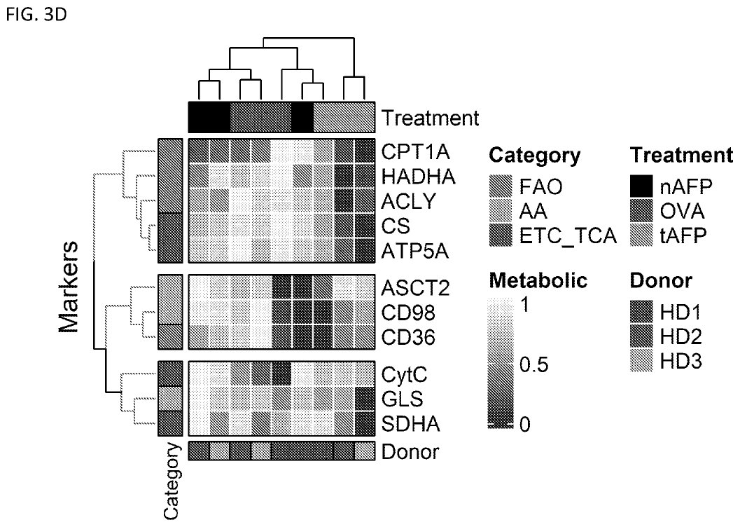

To dissect the metabolic pathways impacted by AFP, we utilized the recently described single-cell metabolic regulome profiling (scMEP, Hartmann et al.) on OVA, nAFP, and tAFP-treated cells. Consistent with a less differentiated phenotype and with our previous studies (Pardee, A. D., Shi, J. & Butterfield, L. H. Tumor-Derived α-Fetoprotein Impairs the Differentiation and T Cell Stimulatory Activity of Human Dendritic Cells. J Immunol. 193, 5723-5732 (2014); (Santos, P. M. et al. Tumor-Derived α-Fetoprotein Suppresses Fatty Acid Metabolism and Oxidative Phosphorylation in Dendritic Cells. Cancer Immunol. Res. 7, 1001-1012 (2019)), tAFP cells had reduced expression of DC markers including CD206, PD-L1, CD11b, CD1c, HLA-DR, CD86 and CD11c (FIG. 3A). Multiple metabolic parameters related to the electron transport chain (ETC)/tricarboxylic acid cycle (TCA), including ATP5A, CS and SDHA, were lower in tAFP treated cells (FIG. 3B). However, levels of cytochrome C (CytC) were elevated in both nAFP and tAFP-treated DCs (FIG. 3B). Fatty acid oxidation (FAO) associated or fatty acid synthase (FAS) proteins CD36, CDPT1A, HADHA, and ACLY were decreased in tAFP-treated mDCs. Proteins involved in amino acid (AA) pathways had modest changes, CD98 and G6PD were significantly decreased and GLS tended to be lower, while ASCT2 was unchanged (FIG. 3B). Proteins involved in glycolysis (GLUT1, GLUT3, LDHA, ENO1, GAPDH, and MTC1) displayed modest differences that were not statistically significant. A schematic summarizing the proteins that were upregulated, unchanged or downregulated is shown (FIG. 3C). A heatmap with hierarchical clustering based on metabolic markers (FAO, AA, and ETC/TCA) and treatment condition (FIG. 3D). The tAFP treated mDC cluster separately from the nAFP or OVA treated mDC. This clustering was due in large part to FAO and ETC/TCA proteins CPT1A, HADHA, ACLY, CS, and ATPSA.

tAFP Induces Transcriptional Changes Consistent with PUFA Exposure

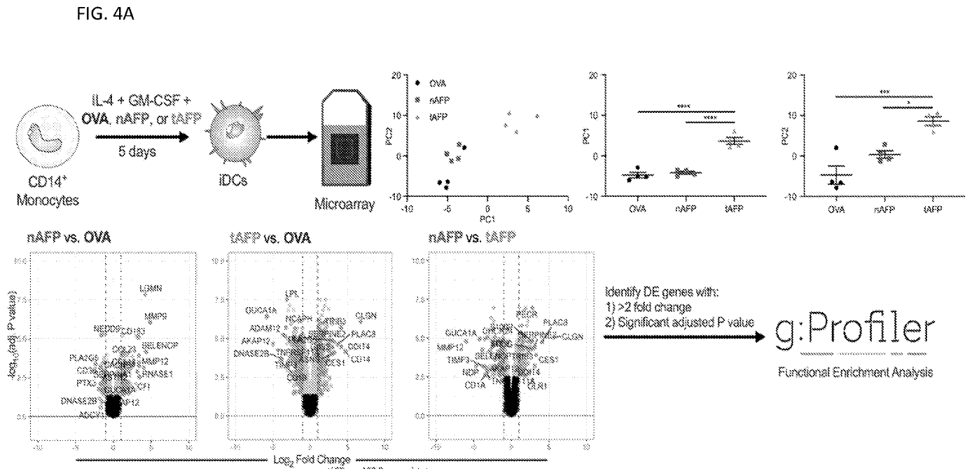

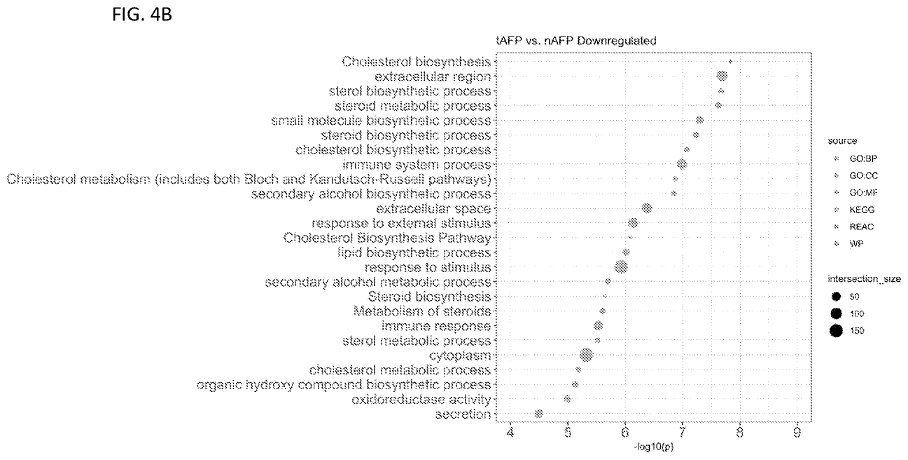

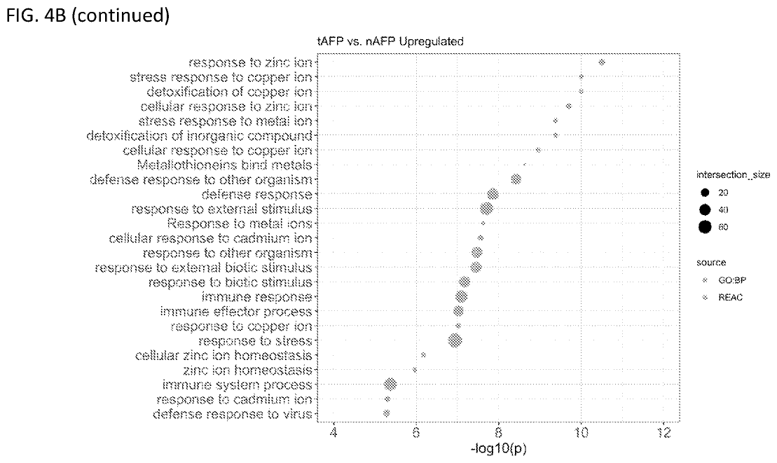

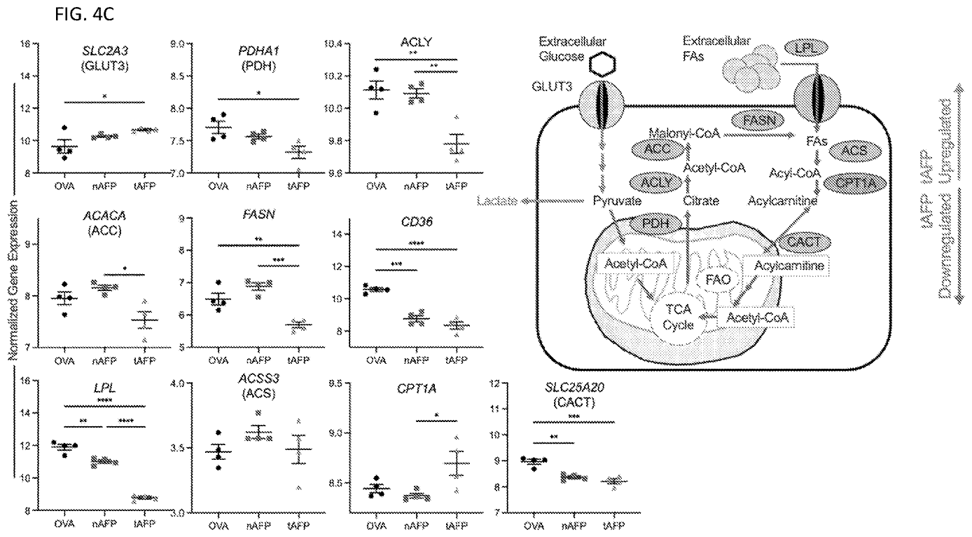

Given the AFP-induced protein level changes in multiple transcription factors, we determined the tAFP induced transcriptional changes in DCs by microarray (FIG. 4). Principle component (PC) analysis revealed tAFP clustered separately from OVA and nAFP, and PC1 and PC2 were independently significant (FIG. 4A). We developed volcano plots for the three possible comparisons to determine differentially expressed genes (DEG). Consistent with the metabolic data, analysis revealed that pathways associated with lipid metabolism were significantly downregulated in tAFP compared to nAFP. The upregulated gene pathways demonstrated a stress response to metal ions, particularly zinc (FIG. 4B).

To dissect these classifications, we analyzed individual genes that could be important in promoting glycolysis and downregulated FA uptake or oxidation. Consistent with tAFP decreasing free glucose in the media and increasing lactate concentrations, we observed an increase in SLC2A3 (GLUT3) gene expression (FIG. 4C). In contrast, we observed a consistent tAFP-induced downregulation with genes involved in fatty acid metabolism, including genes encoding for PDH, ACLY, ACC, FASN, LPL, CD36, and CACT (FIG. 4C). Interestingly, the ACS gene did not reach statistical significance, and tAFP increased the gene expression of CPT1A. Overall, these data provide strong orthogonal evidence in agreement with the SCENITH and scMEP experiments that tAFP alters fatty acid metabolism at the transcript level.

tAFP Ligands are Enriched for Zn and PUFAs.

To further define the mechanism by which tAFP alters metabolism and function of DC (more so than nAFP), we examined the AFP-bound low molar mass (LMM) ligands (Pardee, A. D., Shi, J. & Butterfield. L. H. Tumor-Derived ax-Fetoprotein Impairs the Differentiation and T Cell Stimulatory Activity of Human Dendritic Cells. J Immunol. 193, 5723-5732 (2014)) which we previously demonstrated altered immunoregulatory properties of tAFP (Santos, P. M. et al. Tumor-Derived α-Fetoprotein Suppresses Fatty Acid Metabolism and Oxidative Phosphorylation in Dendritic Cells. Cancer Immunol. Res. 7, 1001-1012 (2019)). nAFP has been previously shown to bind more polyunsaturated fatty acids (PUFAs)³² and zinc (Zn) (Wu, J. T., Monir-Vaghefi, S. M. & Clayton, F. Human alpha-fetoprotein and albumin: differences in zinc binding. Clin. Physiol. Biochem. 5, 85-94 (1987); Permyakov, S. E. et al. Human alpha-fetoprotein as a Zn(2+)-binding protein. Tight cation binding is not accompanied by global changes in protein structure and stability. Biochim. Biophys. Acta 1586, 1-10 (2002)) than albumin. Zn can induce tolerogenic DC (George, M. M., Subramanian Vignesh. K., Landero Figueroa, J. A., Caruso, J. A. & Deepe, G. S. Zinc Induces Dendritic Cell Tolerogenic Phenotype and Skews Regulatory T Cell-Th17 Balance. J Immunol. Baltim. Md 1950 197, 1864-1876 (2016)), and PUFAs are known inhibitors of DC differentiation (Zeyda, M. et al. Polyunsaturated Fatty Acids Block Dendritic Cell Activation and Function Independently of NF-κB Activation. J. Biol. Chem. 280, 14293-14301 (2005)) and have previously been shown to limit lipid metabolism in hepatocytes, in particular through the direct downregulation of the FASN gene. These findings, taken together, suggest that the tAFP may bind more Zn and PUFAs compared to nAFP and therefore result in more potent immuno-metabolic changes.

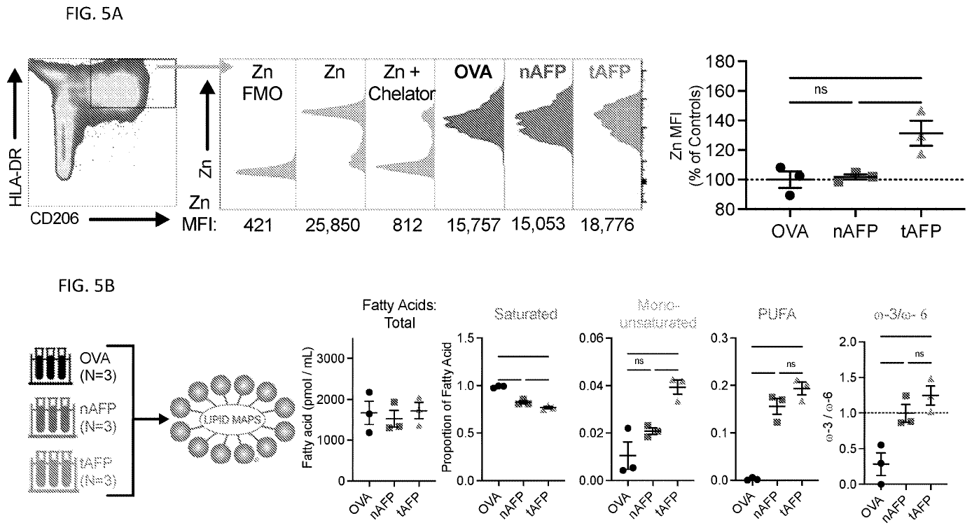

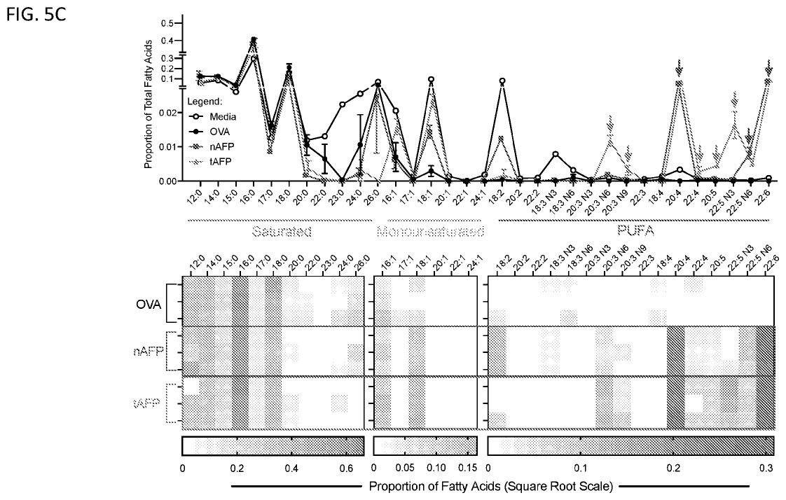

Based on the g:Profiler Zn gene signature (FIG. 4B), we quantified the amount of intracellular Zn in OVA, nAFP and tAFP-treated iDCs. Monocytes were differentiated to iDCs in the presence of OVA, nAFP, or tAFP. The tAFP-treated DC had a statistically significant ˜30% increase in Zn MFI compared to nAFP (P=0.0293) or OVA-treated (P=0.0228) iDCs (FIG. 5A). These findings are consistent the transcriptional data (FIG. 4), and demonstrate that tAFP is more efficient at increasing intracellular Zn concentrations in iDCs, when compared to OVA or nAFP.

OVA, nAFP and tAFP LMM ligands were quantified by mass spectrometry and gas chromatography. The total quantity of bound fatty acids was similar among all proteins, with a mean concentration of approximately 1,500 pmol/mL. In contrast, tAFP bound less saturated fatty acids (SUFAs) (mean=77%) compared to nAFP (82%, P=0.0003) or OVA (99%, P<0.0001). While the amount of monounsaturated fatty acids (MUFAs) was low among all proteins, tAFP bound 2 and 4-fold more MUFAs compared to OVA (P=0.0042) and nAFP (P=0.0318), respectively (FIG. 5B). Polyunsaturated fatty acids (PUFAs) were greater on tAFP (P<0.0001) and nAFP (P=0.0003) when compared to OVA. Based on the terminal double bond location, PUFAs can be further divided into ω-3 and ω-6. Animals cannot convert between ω-3 and ω-6 PUFAs, and they have unique immunological properties, with ω-3 tending to be more potently immunoregulatory—although there is evidence of DC inhibition by ω-6 PUFAs. Interestingly, both tAFP (P=0.0067) and nAFP (P=0.0264) had greater ω-3:ω-6 ratios than OVA. Next, we examined each protein's ligand composition based upon the carbon length and the number of double bonds of each FA. While we did not observe a bias based on FA length, nAFP and tAFP tended to bind FAs with 4 or more double bonds. Next, we analyzed the proportion of individual FAs from each protein (FIG. 5C). We hypothesized that FAs present in high quantities the media would be unlikely to mediate tAFP's immunoregulatory properties, and FAs unique to tAFP would be compelling candidates. To identify FAs statistically unique to tAFP, we generated volcano plots for all three possible comparisons (FIG. 5D). Only a single fatty acid, 17:0, was increased on OVA. As expected, both nAFP and tAFP bound several PUFAs at a greater concentration relative to OVA. When comparing nAFP and tAFP, PUFAs were enriched on nAFP (18:2, 18:3 N3) and tAFP (16:1, 20:3 N6, 22:5 N3, 20:5). To determine which were shared or unique based on each comparison, a Euler diagram of all the differentially bound fatty acids was generated (FIG. 5E). Several of these differentially bound FAs were present in the media (18:2, 18:3 N3, 16:1, 18:1) or attached equally to nAFP and tAFP (20:4, 22:6, 22:5N6). However, the three fatty acids 20:5, 20:3 N6, 22:5N3 were statistically increased on tAFP and not present in the other comparisons.

PUFA Restore tAFP's Suppression of DCs

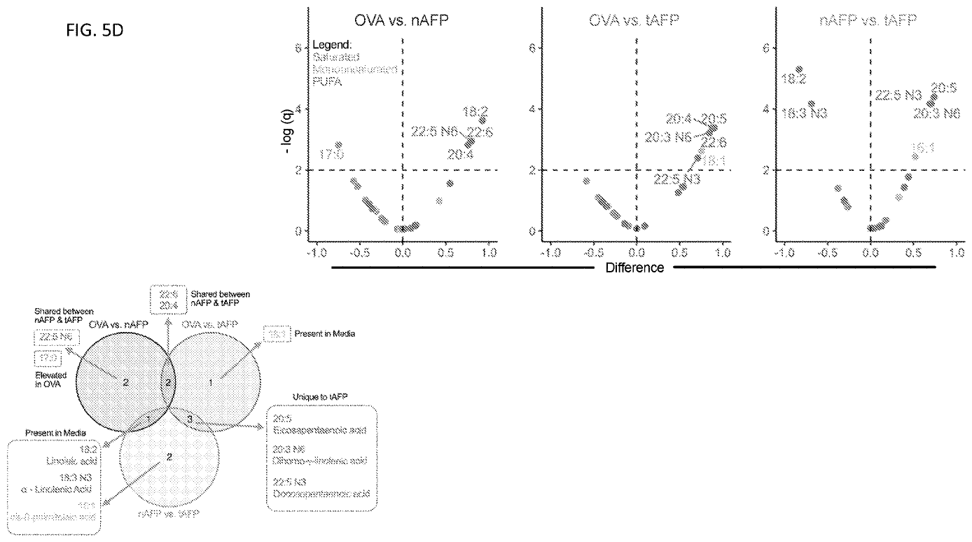

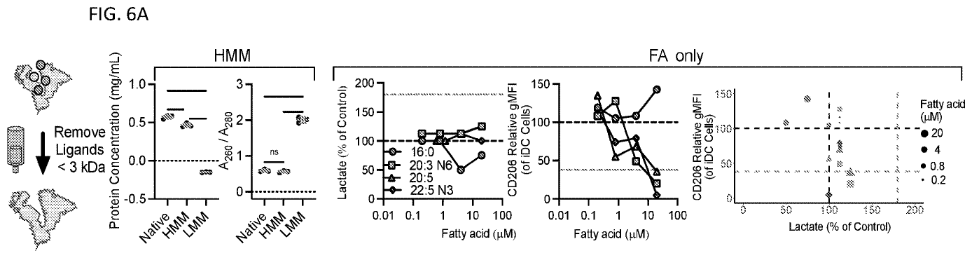

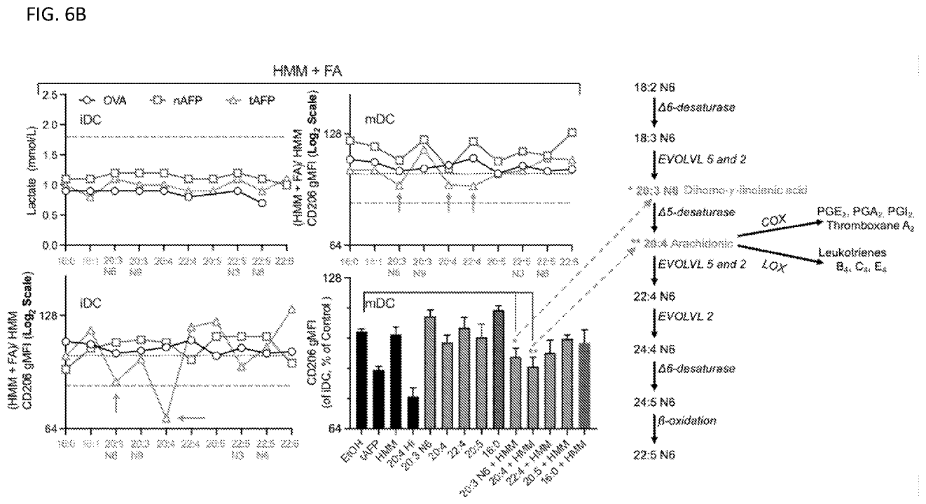

We developed an in vitro assay to screen specific FAs that are necessary for tAFP-mediated suppression of DC formation (FIG. 6A). Many of the FAs screened have known roles in promoting (Weatherill, A. R. et al. Saturated and Polyunsaturated Fatty Acids Reciprocally Modulate Dendritic Cell Functions Mediated through TLR4. J Immunol. 174, 5390-5397 (2005); Lancaster, G. I. et al. Evidence that TLR4 Is Not a Receptor for Saturated Fatty Acids but Mediates Lipid-Induced Inflammation by Reprogramming Macrophage Metabolism. Cell Metab. 27, 1096-1110.e5 (2018)) or limiting DC differentiation (Zeyda, M. et al. Polyunsaturated Fatty Acids Block Dendritic Cell Activation and Function Independently of NF-κB Activation. J Biol. Chem. 280, 14293-14301 (2005)). Therefore, to determine their necessity for tAFP-mediated DC suppression, we titrated several FAs unique to tAFP (20:3 N6, 20:5, and 22:5 N3), and other PUFAs to determine the concentration they lost their inherent ability to suppress DC differentiation in the absence of OVA, nAFP, or tAFP (FIG. 6A). None of the FAs, at any concentration, tested induced production of lactate to levels caused by tAFP indicating that lactate secretion and reduced costimulatory molecule expression are separable immune suppression effects. Treatment with high concentrations (5-20 μM) of 16:0 (palmitic acid) tended to decrease the production of lactate relative to control cells (black-dashed line), suggesting a less glycolytic phenotype (FIG. 6B). All three PUFAs at high concentrations inhibited CD206 expression on DCs (Zeyda. M. et al. Polyunsaturated Fatty Acids Block Dendritic Cell Activation and Function Independently of NF-κB Activation. J Biol. Chem. 280, 14293-14301 (2005)), at levels equivalent to or greater than tAFP treatment. In contrast, the saturated fatty acid 16:0 (palmitic acid) tended to promote DC differentiation (Weatherill, A. R. et al. Saturated and Polyunsaturated Fatty Acids Reciprocally Modulate Dendritic Cell Functions Mediated through TLR4. J Immunol. 174, 5390-5397 (2005)). All PUFAs lost immunoregulatory activity at the 0.2 μM concentration (FIG. 6B).

While all of the PUFAs could inhibit CD206 expression of DC, they did not robustly increase lactate production under these conditions; in contrast, the saturated FA palmitic acid was unique in its ability to increase CD206 expression and decrease lactate secretion (FIG. 4D). None of the FAs combined with either OVA, nAFP or tAFP induced lactate secretion comparable to native tAFP. When measuring CD206 expression at the iDC stage, we observed immunoregulatory activity with FAs 20:3 N6 and 20:4 when combined with tAFP—but not with OVA or nAFP (FIG. 6A). For mDC, we observed inhibition (˜-15%) of CD206 expression with 20:3 N6, 20:4, and 22:4 when combined with tAFP (FIG. 6B). The more modest reduction at the mDC timepoint suggests that treatment with rIFN-γ and LPS can partially, but not entirely reverse, the effects of HMM tAFP+PUFAs. When various FA+HMM tAFP were compared to controls, only 20:3N6 and 20:4 showed significantly reduced CD206 expression (FIG. 6B). Importantly, in the metabolism of ω-6 FAs, 20:3 N6 is converted to 20:4, which can then be further converted into a variety of molecules by COX and LOX enzymes (FIG. 6B). Taken together, these data suggest that PUFAs are necessary for tAFP inhibition of DC differentiation in vitro. Furthermore, these effects may result from exposure to increased 20:3 N6 and/or 20:4, COX/LOX enzymatic derivates of 20:4.

### HCC Patient Monocyte and DC Metabolic Profiling

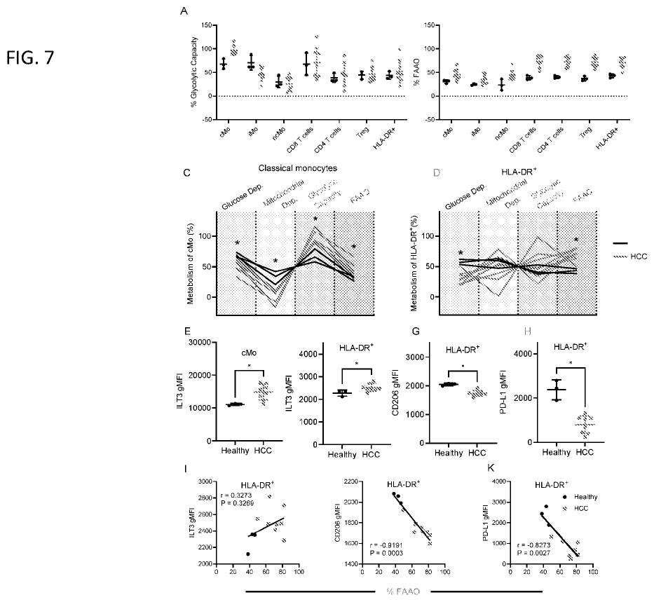

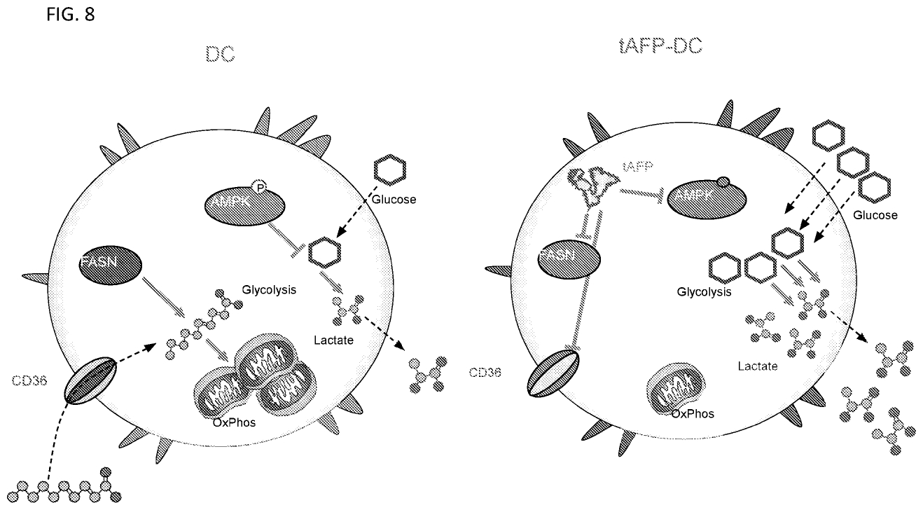

To better understand how these in vitro results extend to in vivo circulating patient myeloid cells, we measured the immunometabolic profile of HCC patient and healthy donor (HD) peripheral blood mononuclear cells (PBMC). SCENITH was used to determine the % glycolytic capacity and FAAO (FIG. 7A, B) across cell types. Monocytes were classified as classical (CD14⁺CD16⁻, cMo), intermediate (CD14⁺CD16⁺, iMo) or non-classical (CD14⁻CD16⁺, ncMo). Among the monocyte subsets, metabolic differences between HCC and HD were most consistent among the cMo (FIG. 7A, B). cMo from HCC patients had decreased glucose and mitochondrial dependence and increased glycolytic capacity and FAAO compared to HD (FIG. 7C). Patient-derived classical monocytes resembled in vitro differentiated DCs treated with AFP (FIG. 1B), showing decreased mitochondrial dependency and elevated glycolytic capacity. In contrast to the in vitro generated DCs, HCC patients had decreased glucose dependence and increased FAAO (FIG. 7D). These in vivo findings are in partial contrast with the prior in vitro data that suggested AFP-treated DCs were more glucose-dependent and had decreased FAAO (FIG. 1B). Regardless of the source of DCs, both in vitro treatment with tAFP or ex vivo derived from HCC patients was associated with DCs retaining a more monocyte-like metabolic phenotype, consistent with tAFP impairing the immuno-metabolic reprogramming required for generating immunostimulatory DCs.

Given the correlation between metabolic state and suppression of key stimulatory molecules (FIG. 2), we measured immune markers on cMo and DCs. The immunoglobulin-like transcript 3 (ILT3) is an important inhibitory receptor expressed on multiple myeloid cells, including monocytes and DCs (Cella, M. et al. A novel inhibitory receptor (ILT3) expressed on monocytes, macrophages, and dendritic cells involved in antigen processing. J. Exp. Med. 185, 1743-1751 (1997). Manavalan, J. S. et al. High expression of ILT3 and ILT4 is a general feature of tolerogenic dendritic cells. Transpl. Immunol. 11, 245-258 (2003); Chang, C. C. et al. Tolerization of dendritic cells by T(S) cells: the crucial role of inhibitory receptors ILT3 and ILT4. Nat. Immunol. 3, 237-243 (2002)) Consistent with a more immunoregulatory phenotype, HCC cMo (FIG. 7E) and DCs (FIG. 7F) expressed more ILT3 compared to HDs. Consistent with the in vitro data, CD206 was decreased in HCC patient DCs. Interestingly, and in contrast with the in vitro data, HCC DCs expressed less PD-L1 (FIG. 7H). While this may suggest a less immunoregulatory phenotype because iDCs tend to express less PD-L1 than mDCs, it could also result from a blockade in full DC differentiation (Pen, J. J. et al. Interference with PD-L1/PD-1 co-stimulation during antigen presentation enhances the multifunctionality of antigen-specific T cells. Gene Ther. 21, 262-271 (2014)). To understand the connection between these immune markers and their metabolic profiles, we performed correlations between these immune markers on DCs and the % FAAO. While HCC patients DCs tended to have greater % FAAO and ILT3 expression these variables were not significantly correlated (P=0.3269, r=0.3273, FIG. 7I).

The serum concentration of AFP in patient blood (Table 1) did not show a direct correlation with circulating myeloid cell phenotypes or metabolic profiles (not shown). This may indicate that the type or amount of tumor-associated ligands binding tAFP in vivo vary between patients and tumors. This was not entirely unexpected, as our previous clinical trial analyses of DC vaccination, and of NK, CD8+ and CD4+ T cell activity in AFP+ and AFP-HCC patients showed significant skewing and dysfunction which was not directly correlated to in vivo serum AFP concentrations (refs 15-18, add Liu, LB JI '06, Evdokimova, LB JIT 2007, Evdokimova, LB EOBT '08). Also, the tumor microenvironment concentrations of AFP may be different than circulating concentrations.

### Discussion:

Here, using single-cell metabolic profiling, we have identified key metabolic pathways and transcriptional regulation molecules that AFP⁺ HCC employ to suppress DC function. nAFP modestly increases glucose uptake and glucose dependent metabolism and similarly reduces FAO. tAFP has a much more potent impact on DC metabolism, promoting a complete dysregulation of all measured metabolic pathways. tAFP-exposed DC take up more glucose and secrete high levels of lactate, which is a well-recognized immune suppressive mediator (Certo, M., Tsai, C.-H., Pucino, V., Ho, P.-C. & Mauro, C. Lactate modulation of immune responses in inflammatory versus tumour microenvironments. Nat. Rev. Immunol. 21, 151-161 (2021); de la Cruz-López, K. G., Castro-Muñoz, L. J., Reyes-Hernandez, D. O., Garcia-Carrancá, A. & Manzo-Merino, J. Lactate in the Regulation of Tumor Microenvironment and Therapeutic Approaches. Front. Oncol. 9, 1143 (2019); Hirschhaeuser, F., Sattler, U. G. A. & Mueller-Klieser, W. Lactate: a metabolic key player in cancer. Cancer Res. 71, 6921-6925 (2011)). We recently showed that monocytes cultured in vitamin D3 to become functionally tolerogenic also have increased reliance on glycolysis and secrete high levels of lactate (Adamik, 2022). Lactate blockade reversed the immune suppressive phenotype of the tolerogenic DC. Here, we show that tAFP has a similar effect on DC.

We have also identified specific FA binding partners of AFP that mediate some of these effects. These findings are consistent with groups that have shown that PUFAs inhibit DCs, some of these have been previously described (DPA, AA, EPA) while others are newly described here (Dihomo-gamma). We also found consistent findings that palmitic acid can promote DC differentiation and are the first to observe that palmitic acid can promote ox-phos. In our examination of the transcriptional pathways associated with Zn, our findings are consistent with other groups that have shown Zn can induce tolerogenic DC. We now show that tAFP delivers more Zn intracellularly than nAFP and induces a glycolytic phenotype in DCs. While AFP has been known to bind Zn, here we report that the Zn bound to tAFP is important for the observed glycolytic switch.

It is important to consider is that metabolism of in vitro cultured DC may not fully reflect cellular metabolism in circulating cells in vivo, given the high concentration of glucose commonly present in media. Our comparative SCENITH analysis of HD and HCC PBMC revealed DCs that more closely resembled the metabolic profile of monocytes than of DCs. Both in vitro-generated DCs treated with tAFP and ex vivo DCs from HCC patients had decreased mitochondrial dependency and increased glycolytic capacity when compared to controls. However, though in vitro generated DCs had increased glucose dependency and decreased FAAO compared to controls, we saw the opposite pattern in HCC-derived DCs. Despite the similarities and differences between the in vitro and ex vivo DCs, in both instances, DCs treated with tAFP or derived from HCC patients more closely resembled their respective monocyte metabolic profiles. These findings are consistent with tAFP limiting the immune-metabolic reprogramming during monocyte differentiation yielding DCs retaining monocyte profiles. Of note, there are several drugs (including TPST 1120 ref) being studied in HCC to inhibit PPARa and FAO. Such an approach could negatively impact the myeloid compartment and immune reactivity while targeting metabolic dysfunction in tumor cells, which could be investigated.

These data provide mechanistic insights on how AFP antagonizes the innate immune response to limit anti-tumor immunity in vivo. Understanding the impact of tAFP on the tumor immune microenvironment may inform the development of future immune checkpoint inhibition combination strategies in HCC overall and in the subset of patients with high tumor AFP expression. Furthermore, these data suggest novel strategies to generate more potent DC vaccines for HCC patients including supplementing culture media with saturated fatty acids and inclusion of Zn chelators.

### Methods:

### Patient Samples

HCC patient (Table 1) and healthy donor (HD) blood (purchased (Trima Residuals RE202, Vitalant)) was collected in BD Vacutainer™ heparin tubes (Cat #02-689-6), and in some cases, BD Vacutainer™ serum tubes (Cat #B-D367820Z) were collected. Heparinized blood was centrifuged to separate the blood and plasma components. Plasma was stored at −80° C. The remaining cellular fraction was overlaid over Ficoll (Cytiva, Cat #45-001-749) in Leucosep™ tubes (Greiner, Cat #07-000-983) and centrifuged to isolate PBMC. PBMC were washed with PBS, and viable cells were quantified via trypan blue (Gibco, Cat #15-250-061) on a Nexcelom Cellometer Spectrum. If cell pellets had substantial red blood cells, they were briefly lysed using ACK lysing buffer (ThermoScientific™, Cat #A1049201). Cells were resuspended in freezing media (80% CellGenix+20% DMSO (MP Biomedicals, Cat #ICN19141880), stored at −80° C. overnight, and stored in gas-phase LN₂.

### In Vitro DC Differentiation

DCs were differentiated in vitro similarly as previously described (Pardee, A. D., Shi, J. & Butterfield, L. H. Tumor-Derived α-Fetoprotein Impairs the Differentiation and T Cell Stimulatory Activity of Human Dendritic Cells. J Immunol. 193, 5723-5732 (2014)). In brief, cryopreserved PBMC were thawed and CD14*monocytes were magnetically labeled using CD14 MicroBeads (Miltenyi, Cat #130-050-201) and isolated by LS columns (Miltenyi, Cat #130-042-401) per the manufacturer instructions. Viable eluted cells were enumerated using trypan blue on a Nexcelom Cellometer Spectrum. To generate iDCs, monocytes were stimulated for 5 days in the presence of 800 IU/mL of rGM-CSF (Miltenyi, Cat #130-093-862) and 500 IU/mL of rIL-4 (Miltenyi, Cat #130-095-373) as well as OVA, nAFP, or tAFP in CellGenix® GMP DC media (Cat #20801-0500) 37° C. at 5% CO2. Lastly, we performed an additional 24-hour stimulation with 1000 IU/mL of rIFN-γ (Peprotech, Cat #300-02) and 250 ng/mL of LPS (Sigma Aldrich, Cat #L2630-10MG) to produce mDCs. To harvest cells, DC were detached using TrypLE™ Select (Gibco, Cat #12563011) for 15 minutes at 37° C. and then washed several times with cold PBS.

### SCENITH

SCENITH was performed as described in (Arguello et al., 2020). SCENITH™ reagents kit (inhibitors, puromycin and antibodies) were obtained from www.scenith.com/try-it and used according to the provided protocol for in-vitro derived myeloid cells. Briefly, control and tol-moDC cultures at desired timepoints, were treated for 18 minutes with Control (DMSO), 2-Deoxy-Glucose (2-DG; 100 mM), Oligomycin (0; 1 μM), a combination of 2DG and Oligomycin (DGO) or Harringtonine (H; 2 μg/mL). Following metabolic inhibitors, Puromycin (final concentration 10 μg/mL) was added to cultures for 17 min. After puromycin treatment, cells were detached from wells using TypLE Select (Fisher Scientific, 505914419), washed in cold PBS and stained with a combination of Human TureStain FcX (Biolegend, 422301) and fluorescent cell viability dye (Biolegend, 423105) for 10 min 4° C. in PBS. Following PBS wash step, primary antibodies against surface markers were incubated for 25 min at 4° C. in Brilliant Stain Buffer (BD Biosciences, 563794). Next cells were fixed and permeabilized using True-Nuclear Transcription Factor Buffer Set (Biolegend, 424401) as per manufacturer instructions. Intracellular staining of puromycin and protein targets was performed for 1 h in diluted (10×) permeabilization buffer at 4° C. Finally, data acquisition was performed using the Cytek Aurora flow cytometer. Primary conjugated antibody information used in SCENITH panel is listed in supplementary table 1. All antibodies were titrated to reduce spillover and increase resolution using single stained moDC (generated as described above) samples. Unstained cell controls used for autofluorescence extraction were generated for each time point, culture conditions (control, vitd3-tol-moDC and dexa-vitd3-tol-moDC) and metabolic inhibitor treatments (C, 2DG, O, DGO). Samples were unmixed using reference controls generated in combination with stained Ultracomp beads (Fisher Scientific, 01-2222-41) and stained cells using the SpectroFlo Software v2.2.0.1. The unmixed FCS files were used for data processing and analysis using FlowJo (BD, version 10.7.1). Manually gated CD14-HLA-DR*CD86+ cells were used for downstream analysis. gMFI expression values were imported into R environment for correlation and heatmap analysis using the below described R packages.

### Glucose and Lactate Measurements

Glucose and lactate were measured by applying approximately 5 μL of supernatant to Clarity BG1000 Blood Glucose strips (Cat #75840-798) and meter (Cat #75840-800) system or the Lactate Plus strips (Nova Biomedical©, Cat #40813) and meter version 2 (Nova Biomedical©, Cat #62624) system. Each meter was quality checked with control glucose and lactate solutions and CellGenix™ media before each experiment.

### CyTOF Phenotypic Profiling

scMEP analysis was performed as recently described (Hartmann et al., 2021). In short, antibodies targeting metabolic features were conjugated in-house using an optimized conjugation protocol (Hartmann et al., 2019) and validated on multiple sample types. Cells were prepared for scMEP analysis by incubation with small molecules to be able to assess biosynthesis rates of DNA, RNA and protein, cisplatin-based live/dead staining, PFA-based cell fixation and cryopreservation (dx.doi.org/10.17504/protocols.io.bkwkkxcw). Next, cells were stained with metabolic antibodies in a procedure that includes surface staining for 30 min at RT, PFA-fixation for 10 min at RT, MeOH-based permeabilization for 10 min on ice, intracellular staining for 1 h at RT and DNA intercalation (dx.doi.org/10.17504/protocols.io.bntnmeme). Finally, cells were acquired on a CyTOF2 mass cytometer (Fluidigm). Protein targets and antibody information used in scMEP are listed in supplementary table 2. Raw mass spectrometry data were pre-processed, de-barcoded and imported into R environment using the flowCore package (version 2.0.1) (Hahne et al., 2009). Values were arcsin h transformed (cofactor 5) and normalized (Hartmann et al., 2021) for downstream analyses based on previously reported workflow (Nowicka et al., 2017).

Microarray and gProfiler

OVA, nAFP, and tAFP-treated DC were lysed, and total mRNA was obtained for microarray (Affymetrix HG-U133A). DE genes were uploaded in g:Profiler in R Studio for pathway analysis and visualization (Raudvere, U. et al. g:Profiler: a web server for functional enrichment analysis and conversions of gene lists (2019 update). Nucleic Acids Res. 47, W191-W198 (2019)).

### Zn Measurement

Intracellular Zn was quantified by flow cytometry using the Zinc Assay Kit (Cell-based) (Abcam, Cat #ab241014). Monocytes were differentiated to iDCs as described above in the presence of OVA, nAFP, tAFP, or ZnSO₄. Zn staining was performed per manufacturer suggested protocol with positive (Zn) and negative (Zn+chelator) controls as well as a Zn FMO included in each experiment. Cells were stained with LD Aqua for 10 minutes at RT. Cells were washed in 1× Assay Buffer, then stained in 100 μL of Assay Buffer+0.2 μL of Zn Probe for 30 minutes at 37° C. Cells were then washed twice with 1× Assay buffer then stained with HLA-DR-APC-H7 (BD, Clone: GF6-6, Cat #561358, Lot #0023290, 0339025), CD86-BV785 (BioLegend, Clone: IT2.2, Cat #305441, Lot #B277560), CD206 PE-Cy7 (Biolegend, Clone: 15-2, Cat #321123, Lot #B331254), and CD14-BUV805 (BD, Clone: M5E2, Cat #612903, Lot #0297714), in Brilliant Stain Buffer (BD Horizon, Cat #566349, Lot #0121427) for 20 minutes at 4° C. Cells were washed twice in FACS Buffer and fixed in 1% paraformaldehyde (Thermo Scientific, Cat #J19943-K2, Lot #195273, diluted in PBS) for at least 30 minutes before acquisition on a BD LSRFortessam X-50. As a negative control we briefly treated cells with Zn but did not stain for Zn as a fluorescence-minus-one (FMO) control (MFI=421) or stained with a Zn probe as a positive control (MFI=25,850). Zn treated cells were treated with a Zn chelator included in the kit before staining, and this resulted in a marked ˜₉₇% reduction in Zn MFI compared to the positive control.

### Lipid Analysis by Mass Spectrometry or Gas Chromatography

Commercially available OVA (N=3) (Sigma Aldrich, Cat #A5503-1G, Lot #SLCB8249), nAFP (N=3) (Cell Sciences, Cat #CSI10379, Lot #4111714), and tAFP (N=3) (Bio-Rad, Cat #13752600, Lot #64110896) were submitted diluted in PBS (Gibco™, Cat #20-012-050) at 1000 ug/mL on dry ice. CellGenix® GMP Dendritic Cell Medium (N=1) (CellGenix®, Cat #20801-0500) media and supernatants of mDCs from a healthy donor (N=1) differentiated in the presence of 5 μg per mL of OVA, nAFP, or tAFP were tested. Lipid analysis (Supplementary Table 1) was performed at the UCSD Lipidomics Core (Quehenberger, O. et al. Lipidomics reveals a remarkable diversity of lipids in human plasma. J Lipid Res. 51, 3299-3305 (2010)).

### Fatty Acid Screen

Fatty acids (Supplementary Table 3) were acquired from Cayman Chemical (Ann Arbor, MI, USA) including 16:0 (palmitic acid, Item #10006627, Batch #0523612-48), 18:1 (oleic acid, Item #90260, Batch #0540276-62), 20:3 N6 (dihomo-γ-linolenic acid, Item #90230, Batch #0532009-37), 20:3 N9 (5,8,11-eicosatrienoic acid, Item #90190, Batch #0564724-7), 20:4 (arachidonic acid, Item #90010, Batch #0570304-50), 22:4 (adrenic acid, Item #90300, Batch #0537603-20), 20:5 (eicosapentaenoic acid, Item #26415, Batch #0583627), 22:5 N3 (docosapentaenoic acid, Item #90165, Batch #0569492-11), 22:5 N6 (docosapentaenoic acid, Item #10008335, Batch #0462864-36), and 22:6 (docosahexaenoic acid, Item #90310, Batch #0593448-15).

Fatty acids were resuspended in ethanol and stored at −20° C. at 100 uM. High molar mass (HMM) fractions of OVA, nAFP, and tAFP were obtained by removing the low molar mass (LMM) contents with the Amicon® Ultra—0.5 mL Centrifugal Filters Ultracel®—3K (Millipore, Cat #UFC500324, Lot #R9HA51100) per the manufacturer suggested protocol and stored at −80° C. Both the native preparations and HMM fractions contained similar amounts of protein (˜0.5 mg/mL), whereas protein was undetectable (<0 mg/mL) in the low molar mass (LMM) fraction. Additionally, we determined the A₂₆₀/A₂₈₀ ratio as a measure of purity and found the LMM fraction had a ˜3-fold increase in the A₂₆₀/A₂₈₀ ratio indicating a large proportion of non-protein compounds in the LMM fraction, as expected. Fats (+/−HMM) were added to pre-warmed media and incubated for 1 hour, mixing at 37° C. before adding to cells (Alsabeeh, N., Chausse, B., Kakimoto, P. A., Kowaltowski, A. J. & Shirihai, O. Cell Culture Models of Fatty Acid Overload: Problems and Solutions. Biochim. Biophys. Acta 1863, 143-151 (2018)). Fats were combined with HMM at a 3:1 molar ratio, as previously described (Alsabeeh, N., Chausse, B., Kakimoto, P. A., Kowaltowski, A. J. & Shirihai. O. Cell Culture Models of Fatty Acid Overload: Problems and Solutions. Biochim. Biophys. Acta 1863, 143-151 (2018)).

### Statistics and Visualization

Statistical comparisons between groups were performed using paired-sample t-tests unless otherwise stated using R (version 4.0.2) and R Studio (Version 1.3.1093) or Prism (Version 9.0.2). P values are represented as *p≤0.05, **p≤0.01, ***p≤0.001, ****p≤0.0001. p values<0.05 were considered statistically significant. Numerical labels indicate near significant values). Figure graphs were generated using the R package ggplot2 (version 3.3.3) or in Prism.

### Study Approval

Blood collection from HCC Patients was approved by the UCSF Hepatobiliary Tissue Bank and Registry Oversight Committee (CC #124512). The UCSF Cancer Immunotherapeutics Tissue Use Committee approved samples from healthy donors at UCSF (CC #16983).

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A pharmaceutical composition comprising (i) alpha fetoprotein (AFP) and (ii) dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, or leukotriene E₄.

2. The pharmaceutical of claim 1, wherein the AFP is tumor AFP (tAFP) or normal AFP (nAFP).

3. A method of reducing an inflammatory response in a human in need thereof, the method comprising administering a composition comprising a sufficient amount of alpha fetoprotein (AFP) bound to dihomo-gamma-linolenic acid (20:3, n-6)-, arachidonic acid (20:4)-, prostaglandin E₂ (PGE₂)-, prostaglandin A₂ (PGA₂)-, prostacyclin (PGl₂)-, thromboxane A₂-, leukotriene B₄-, leukotriene C₄-, or leukotriene E₄-bound alpha fetoprotein (AFP) thereby reducing the inflammatory response.

4. The method of claim 3, wherein the AFP is tumor AFP (tAFP) or normal AFP (nAFP).

5. The method of claim 4, wherein the human has an autoimmune disease.

6. The method of claim 4, wherein the human has Addison disease, celiac disease, dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, or Type I diabetes.

7. A method of making the pharmaceutical composition of claim 1, the method comprising
providing recombinant or purified alpha fetoprotein (AFP); and
mixing the AFP with a fatty acid selected from the group consisting of dihomo-gamma-linolenic acid (20:3, n-6), arachidonic acid (20:4), prostaglandin E₂ (PGE₂), prostaglandin A₂ (PGA₂), prostacyclin (PGl₂), thromboxane A₂, leukotriene B₄, leukotriene C₄, and leukotriene E₄ to generate dihomo-gamma-linolenic acid (20:3, n-6)-, arachidonic acid (20:4)-, prostaglandin E₂ (PGE₂)-, prostaglandin A₂ (PGA₂)-, prostacyclin (PGl₂)-, thromboxane A₂-, leukotriene B₄-, leukotriene C₄-, or leukotriene E₄-bound AFP.

8. The method of claim 7, wherein the mixing comprises forming a mixture of the tAFP and the fatty acid at a molar ratio of between 1:1 and 1:5.

9. The method of claim 7, wherein the AFP is tumor AFP (tAFP) or normal AFP (nAFP) or synthetic AFP.

10. A method of preparing a dendritic cell vaccine, the method comprising,
culturing dendritic cells in a culture medium comprising palmitic acid-bound tumor alpha fetoprotein (tAFP) under conditions to generate mature dendritic cells expressing tAFP-derived HLA-restricted peptides.

11. The method of claim 10, wherein the culturing occurs in in the absence of other polyunsaturated fatty acids.

12. The method of any one of claims 10-12, wherein the culture medium comprises a zinc chelator.

13. The method of claim 10, wherein the dendritic cells are immature dendritic cells.

14. The method of claim 10, wherein the dendritic cells are mature dendritic cells.

15. A dendritic cell vaccine comprising mature dendritic cells cultured in the presence of palmitic acid-bound alpha fetoprotein (AFP) in the absence of other polyunsaturated fatty acids, wherein the mature dendritic cells express AFP-derived HLA-restricted peptides.

16. The dendritic cell vaccine of claim 15, wherein the AFP is tumor AFP (tAFP) or normal AFP (nAFP) or synthetic AFP.

17. A method of stimulating an immune response to tAFP in a human, the method comprising,
administering to a human a sufficient amount of the dendritic cell vaccine of claim 15 to stimulate an immune response to tAFP.

18. The method of claim 17, wherein the human has AFP-positive liver cancer.

19. The method of claim 17 or 18, comprising,
obtaining immature dendritic cells from the human,
culturing the immature dendritic cells in a culture medium comprising palmitic acid-bound tumor alpha fetoprotein (tAFP) in the absence of other polyunsaturated fatty acids under conditions to generate mature dendritic cells expressing tAFP-derived HLA-restricted peptides,
and then administering the dendritic cells to the human.

20. The method of claim 19, wherein the culture medium comprises a zinc chelator.

21. A method, comprising,
obtaining a sample from a human having liver cancer; and
measuring the amount of, and identity of, polyunsaturated fatty acids bound to tumor alpha fetoprotein (tAFP) in the sample.
